(19) United States
(12) Patent Application Publication
(10) Pub. No.: US 2013/0005651 A1
(43) Pub. Date: Jan. 3, 2013

(54) DEATH ASSOCIATED PROTEIN 1 VARIANTS AND USE THEREOF FOR MODULATING AUTOPHAGY

(75) Inventors: Adi Kimchi, Rehovot (IL); Itay Koren, Rehovot (IL)

(73) Assignee: YEDA RESEARCH AND DEVELOPMENT CO. LTD., Rehovot (IL)

(21) Appl. No.: 13/583,924

(22) PCT Filed: Mar. 10, 2011
(86) PCT No.: PCT/IL11/00239

§ 371 Date: Sep. 10, 2012

Related U.S. Application Data
(60) Provisional application No. 61/312,684, filed on Mar. 11, 2010.
(60) Provisional application No. 61/348,256, filed on May 26, 2010.

(51) Int. Cl. A61K 38/17; A61P 25/28; A61P 3/10; C12N 1/21; C12N 15/12; C12N 15/63; C12N 5/071; C12N 5/10; A61P 35/00; C07K 14/47

(52) U.S. Cl. 514/6.9; 514/21.2; 514/19.3; 514/17.7; 530/350; 536/23.5; 435/320.1; 435/375; 435/369; 435/367; 435/252.3

(57) ABSTRACT

The present invention relates to methods for the modulation of autophagy by altering the phosphorylation of Death Associated Protein (DAP1). The present invention further relates to methods of treating autophagy associated diseases comprising the suppression of autophagy by dephosphorylating DAP1. The invention further provides human DAP1 mutated at positions 3 and 51 with phospho-silencing residues and uses thereof in treating autophagy associated diseases.

### FIELD OF THE INVENTION

The present invention relates to compositions and methods for the modulation of autophagy, useful in promoting or preventing autophagy in target cell populations. In particular, the invention relates to use of Death Associated Protein 1 (DAP1) and variants thereof for modulating autophagy, thereby treating autophagy associated diseases.

### BACKGROUND OF THE INVENTION

Autophagy, a catabolic process responsible for the degradation of cytosolic components, is upregulated when nutrient supplies are limited (Yang, Z. and Klionsky, D. J. (2010), Curr. Opin. Cell Biol., 22:124-31). Autophagy is characterized by the formation of double membrane enclosed autophagosomes which engulf intracellular organelles and cytoplasmic constituents, and deliver them to the lysosomes for degradation. In addition to its cytoprotective functions in stressed cells (Levine and Kroemer, 2008, Cell, 132, 27-42), autophagy can serve as a cell death mechanism under some conditions (Berry and Baehrecke, 2007, Cell, 131, 1137-48). Recent studies have demonstrated that autophagy is closely related to the occurrence and development of numerous pathological processes, including myopathy, neurodegenerative disorders, tuberculosis, cancer, type II diabetes and others.

A critical step in the induction of autophagy comprises the inactivation of a key negative regulator of the process, the Ser/Thr kinase mammalian target of rapamycin (mTOR) (Laplante, M. and Sabatini, D. M., (2009), J. Cell Sci., 122:3589-94). Thus far, only a few direct substrates of mTOR which control autophagy have been identified, such as ULK1 and Atg13 which function as positive mediators.

Some preliminary documentation of the involvement of DAP1 in autophagy was recently provided in a planarian remodeling system, in which expression of the planarian DAP1 orthologue is activated in the population of cells undergoing autophagy during the remodeling/regeneration process (Gonzalez-Estevez, C., et. al., (2007), PNAS, 104:13373-8). To date, little is known about suppressors of autophagy and only a few have been identified, including mTOR and several members of the Bcl-2 family (Maiuri, M. C., et. al., (2007), Embo J., 26:2527-39; Pattingre S., et. al., (2005), Cell, 122:927-39).

International publication application No. WO 95/010630, of one of the inventors of the present invention, relates to, inter alia, DAP DNA molecules, expression vectors comprising them, or DAP products (i.e., expression products of the DAP DNA molecules) for promoting death of normal or tumor cells. The '630 publication also relates to agents which prevent the expression of said DAP DNA molecules, or agents which antagonize, inhibit or neutralize the DAP products, for protecting cells from programmed cell death.

None of the background art, however, discloses or suggests the regulation of autophagy by altering DAP1 phosphorylation, particularly modulating the phosphorylation of serine residues at position 3 and/or 51 of a human DAP1. In addition, none of the background art discloses or suggests that altering DAP1 phosphorylation is useful in treating autophagy associated diseases.

There is an unmeet need for compositions and methods useful in mediating autophagy in target cells, thereby useful in treating autophagy associated diseases such as, cancer and neurodegenerative diseases.

### SUMMARY OF THE INVENTION

The present invention provides compositions and methods for the modulation of autophagy by altering the phosphorylation of Death Associated Protein 1 (DAP1). The compositions and methods of the present invention are useful for promoting or preventing autophagy in target cell populations.

It is disclosed herein for the first time that human DAP1 is a suppressor of autophagy. It is further disclosed for the first time that human DAP1 is functionally silenced when phosphorylated.

The present invention is based in part on the surprising discovery that the knockdown of DAP1 enhanced autophagic flux. In addition, a rapid decline in DAP1 phosphorylation was observed following amino acid starvation. The mapping of the phosphorylation sites indicated that DAP1 is functionally silenced in growing cells through phosphorylations on Ser3 and Ser51. Specifically, the phosphorylation of serine 3 and serine 51 was found to be mTOR-dependent. Furthermore, inactivation of mTOR during starvation, caused a rapid reduction in these phosphorylation sites, and converted DAP1 into an active suppressor of autophagy. Notably, substitution of the serine residues at positions 3 and 51 by aspartic acid residues resulted in the production of a DAP1 variant lacking the ability to suppress autophagy when overexpressed.

Thus, the present invention provides compositions and methods for the modulation of autophagy, particularly by altering the phosphorylation state of DAP1. According to some embodiments, altering DAP1 phosphorylation comprises altering (e.g., enhancing or reducing) phosphorylation of amino acid residues Ser3 and/or Ser51. It is to be emphasized that according to some embodiments, DAP1 is a suppressor of autophagy when Ser3 and/or Ser51 are dephosphorylated. The present invention further provides DAP1 variants wherein at least one serine residue selected form serine 3 and serine 51 of human DAP1 is substituted. In some embodiments, the DAP1 variant comprises serine substitutions with a residue incapable of being phosphorylated (e.g. alanine), useful in suppressing or reducing autophagy. In other embodiments, the DAP1 variant comprises serine substitution with a phspho-mimicking residue, useful in promoting autophagy in a cell.

According to a first aspect, the present invention provides a method for the modulation of autophagy comprising altering the phosphorylation of DAP1. According to one embodiment, said DAP1 is a mammalian DAP1, preferably a human DAP1. According to a specific embodiment, the human DAP1 comprises the amino acid sequence as set forth in SEQ ID NO:1. According to yet another specific embodiment, the human DAP1 consists of the amino acid sequence as set forth in SEQ ID NO: 1. According to another embodiment, altering the phosphorylation of DAP1 comprises altering the phosphorylation state of at least one serine residue selected from serine 3 and serine 51 of human DAP1.

According to some embodiments, modulation of autophagy is an increase in autophagy. According to another embodiment, the method comprises enhancing DAP1 phosphorylation. According to another embodiment, enhancing DAP1 phosphorylation increases or promotes autophagy in said cell. In some embodiments, enhancing DAP1 phosphorylation comprises the phosphorylation of serine 3 and/or serine 51 of said DAP1.

According to another embodiment, modulation of autophagy is a decrease in autophagy. According to another embodiment, the modulation of autophagy is a suppression of autophagy. According to another embodiment, the method comprises reducing DAP1 phosphorylation. According to another embodiment, reducing DAP1 phosphorylation reduces or suppresses autophagy in said cell. In some embodiments, reducing DAP1 phosphorylation comprises reducing phosphorylation (e.g., dephosphorylation) of serine 3 and/or serine 51 of said DAP1.

The terms “Ser3”, “serine 3” and “serine residue at position 3” as used herein refer to the serine reside situated at position 3 of human DAP1 polypeptide (SEQ ID NO:1), wherein the residue at position 1 is the N-terminus of said DAP1 polypeptide.

The terms “Ser51”, “serine 51” and “serine residue at position 51” as used herein refer to the serine reside situated at position 51 of human DAP1 polypeptide (SEQ ID NO:1), wherein the residue at position 1 is the N-terminus of said DAP1 polypeptide.

According to some embodiments, autophagy is reduced or suppressed upon reduction of DAP1 phosphorylation by inactivating the Ser/Thr mammalian target of rapamycin (mTOR). According to some embodiments, the inactivation of mTOR may be performed by any mTOR inhibitor known in the art. Non limiting examples of mTOR inhibitors include the inhibitor compounds described in U.S. Pat. Nos. 7,504,397, 7,659,274 and 7,700,594. Human mTOR is known in the art and in some embodiments has the amino acid sequences as set forth in SEQ ID NO: 12 (NP_{—}004949). According to certain embodiments, autophagy is increased or promoted upon phosphorylation of DAP1 by mTOR, in particular on Ser3 and/or Ser51.

In some embodiments, reducing or suppressing autophagy in a cell comprises reducing or suppressing autophagy in a cancer cell (e.g., a nutrient deprived cancer cell). In additional embodiments, said method for reducing or suppressing autophagy is useful for the treatment of cancer.

According to another aspect, the present invention provides a method for treating an autophagy associated disease or disorder comprising inhibiting or suppressing autophagy in a cell comprising reducing DAP1 phosphorylation. According to another embodiment, DAP1 is a human DAP1 (SEQ ID NO: 1). According to some embodiments, reducing DAP1 phosphorylation comprises reducing the phosphorylation (e.g., dephosphorylation) of at least one serine residue selected from the serine residue at positions 3 or 51 of human DAP1. According to another embodiment, reducing DAP1 phosphorylation comprises reducing the phosphorylation of the serine residue at position 3 of human DAP1. According to another embodiment, reducing DAP1 phosphorylation comprises reducing the phosphorylation of the serine at position 51 of human DAP1. According to another embodiment, reducing DAP1 phosphorylation comprises reducing the phosphorylation of serine at position 3 and serine at position 51 of human DAP1.

According to one embodiment, the autophagy associated disease or disorder is cancer. According to another embodiment, the autophagy associated disease or disorder is a neurodegenerative disease or disorder. According to yet another embodiment, the autophagy related disease or disorder is type II diabetes. According to a further embodiment, the autophagy related disease or disorder is myopathy.

According to some embodiments, the phosphorylation of DAP1 is reduced by inactivating the Ser/Thr mammalian target of rapamycin (mTOR).

According to another aspect, the present invention provides a human DAP1 variant comprising at least one serine residue substituted with a phospho-silencing residue, wherein the at least one serine residue is selected from serine 3 and serine 51 of human DAP1 (SEQ ID NO: 1).

According to one embodiment, the human DAP1 variant comprises a substitution of the serine residues at positions 3 and 51 of human DAP1 with phospho-silencing residues. In a particular embodiment, said human DAP1 variant comprises the amino acid sequence as set forth in SEQ ID NO: 2. In yet another particular embodiment, said human DAP1 variant consists of the amino acid sequence as set forth in SEQ ID NO: 2.

According to another embodiment, the human DAP1 variant comprises a substitution of serine 3 of human DAP1 with a phospho-silencing residue. In a particular embodiment, said human DAP1 variant comprises the amino acid sequence as set forth in SEQ ID NO: 3. In yet another particular embodiment, said human DAP1 variant consists of the amino acid sequence as set forth in SEQ ID NO: 3.

According to another embodiment, the human DAP1 variant comprises a substitution of serine 51 of human DAP1 with a phospho-silencing residue. In a particular embodiment, said human DAP1 variant comprises the amino acid sequence as set forth in SEQ ID NO: 4. In yet another particular embodiment, said human DAP1 variant consists of the amino acid sequence as set forth in SEQ ID NO: 4.

According to one embodiment, the phospho-silencing residue is selected from the group consisting of alanine, isoleucine, leucine, asparagine, lysine, methionine, phenylalanine, glutamine, tryptophan, glycine, valine, proline, arginine and histidine. Each possibility represents a separate embodiment of the invention.

According to some embodiments, the phospho-silencing residue is alanine. According to additional embodiments, the human DAP1 variant comprises the amino acid sequences selected from the group consisting of SEQ ID NO: 13, SEQ ID NO: 14 and SEQ ID NO: 15. In one embodiment, the human DAP1 variant consists of the amino acid as set forth in SEQ ID NO: 13. In another embodiment, the human DAP1 variant consists of the amino acid as set forth in SEQ ID NO: 14. In another embodiment, the human DAP1 variant consists of the amino acid as set forth in SEQ ID NO: 15.

A “phospho-silencing residue” as used herein refers to a residue which is incapable of phosphorylation and is other than a phospho-mimicking residue.

A “phospho-mimicking residue” as used herein refers to a residue which is not phosphorylated but displays physico-chemico properties similar to a residue carrying a phosphate ion (phosphorylated residue) such as for example aspartic acid or glutamic acid.

According to some embodiments, the human DAP1 variants described hereinabove (e.g., SEQ ID NOs: 2-4, and SEQ ID NOs: 13-15) are useful in decreasing or suppressing autophagy in a cell.

According to additional embodiments, the human DAP1 variants described hereinabove (e.g., SEQ ID NOs: 2-4, and SEQ ID NOs: 13-15) are useful in treating autophagy an associated disease or disorder.

According to another aspect, the present invention provides an isolated polynucleotide encoding the human DAP1 variants of the present invention, wherein serine at position 3, or serine at position 51 or both are substituted with a phospho-silencing residue. According to particular embodiments, the isolated polynucleotide encodes a human DAP1 variant selected from the amino acid sequence as set forth in SEQ ID NO: 2-4 and SEQ ID NO: 13-15. Each possibility represents a separate embodiment of the present invention.

According to another embodiment, the present invention provides a recombinant polynucleotide construct wherein a polynucleotide encoding a human DAP1 variant of the present invention is operably linked to a transcription regulating sequence. As known in the art, a transcription regulating sequence can direct the transcription of a polynucleotide in an intended host cell. In another embodiment, the transcription regulating sequence is a transcription initiation sequence.

The invention further provides an expression vector comprising an isolated polynucleotide encoding a human DAP1 variant of the present invention. In another embodiment, there is provided an expression vector comprising a recombinant polynucleotide construct of the invention. According to various embodiments the expression vector is for example, a plasmid or a virus. In some embodiments, the present invention provides a host cell transfected with said expression vector. Typically, the host cell is selected from eukaryotic and prokaryotic cells.

According to another embodiment, the present invention provides a pharmaceutical composition comprising a therapeutically effective amount of an active agent selected from the group consisting of: (a) an isolated polypeptide comprising the amino acid sequence of a human DAP1 variant; (b) an isolated polynucleotide encoding a human DAP1 variant; (c) an expression vector comprising the isolated polynucleotide of (b); and (d) a host cell transfected with the expression vector of (c); further comprising a pharmaceutically acceptable carrier, wherein said human DAP1 variant comprises at least one serine residue selected from serine 3 and serine 51 of human DAP1 (SEQ ID NO: 1) substituted with a phospho-silencing residue.

In some embodiments the pharmaceutical composition comprises as an active agent an isolated polynucleotide encoding a human DAP1 variant of the present invention (e.g., a human DAP1 variant polypeptide having a phospho-silencing residue at positions 3, 51 or both), or an active analog or fragment thereof. In additional embodiments the pharmaceutical composition comprises as an active agent a recombinant polynucleotide construct comprising an isolated polynucleotide encoding a human DAP1 variant of the present invention. According to particular embodiments, said human DAP1 variant is selected from the amino acid sequence as set forth in SEQ ID NO: 2, SEQ ID NO: 3, SEQ ID NO: 4, SEQ ID NO: 13, SEQ ID NO: 14 and SEQ ID NO: 15. Each possibility represents a separate embodiment of the present invention.

According to another embodiment, the present invention provides a method for treating an autophagy associated disease or disorder in a subject in need thereof, comprising administering to the subject a pharmaceutical composition of the present invention (e.g., a human DAP1 variant having a phospho-silencing residue at positions 3, 51 or both), thereby treating the autophagy associated disease or disorder in said subject. According to particular embodiments, said human DAP1 variant is selected from the amino acid sequence as set forth in SEQ ID NO: 2, SEQ ID NO: 3, SEQ ID NO: 4, SEQ ID NO: 13, SEQ ID NO: 14 and SEQ ID NO: 15. Each possibility represents a separate embodiment of the present invention.

According to some embodiments administering to the subject a human DAP1 variant having a phospho-silencing residue at positions 3, 51 or both in cells of the subject reduces or suppresses autophagy, thereby treating an autophagy associated disease or disorder in said subject. Each possibility represents a separate embodiment of the present invention.

According to another embodiment, the method for treating an autophagy associated disease or disorder comprises administering to the subject a therapeutically effective amount of a recombinant polynucleotide construct comprising a polynucleotide encoding the human DAP1 variant having a phospho-silencing residue at positions 3, 51 or both, or an active analog or fragment thereof. According to particular embodiments, said human DAP1 variant is selected from the amino acid sequence as set forth in SEQ ID NO: 2, SEQ ID NO: 3, SEQ ID NO: 4, SEQ ID NO: 13, SEQ ID NO: 14 and SEQ ID NO: 15. Each possibility represents a separate embodiment of the present invention. According to another embodiment, the recombinant polynucleotide construct is introduced into the subject's cells ex vivo. According to another embodiment, the recombinant polynucleotide construct is introduced into the subject's cells in vivo.

An autophagy associated disease or disorder, according to one embodiment, is cancer. According to another embodiment, said autophagy associated disease or disorder is a neurodegenerative disease or disorder. According to yet another embodiment, said autophagy related disease or disorder is type II diabetes. According to a further embodiment, said autophagy related disease or disorder is myopathy.

The present invention further provides a method of selectively reducing or suppressing autophagy in target cells, comprising the step of exposing the target cells to the pharmaceutical composition of the invention (e.g., an isolated polynucleotide encoding a DAP1 variant) in an amount sufficient to reduce or suppress autophagy.

According to another embodiment, the present invention provides a pharmaceutical composition comprising a therapeutically effective amount of an active agent selected from the group consisting of: (a) an isolated polypeptide comprising the amino acid sequence of human DAP1 variants of the present invention, or an active analog or fragment thereof; (b) an isolated polynucleotide encoding a human DAP1 variant, or an active analog or fragment thereof; (c) an expression vector comprising the isolated polynucleotide of (b); and (d) a host cell transfected with the expression vector of (c); further comprising a pharmaceutically acceptable carrier, for use in treating an autophagy associated disease or disorder. According to particular embodiments, said human DAP1 variant is selected from the amino acid sequence as set forth in SEQ ID NO: 2, SEQ ID NO: 3, SEQ ID NO: 4, SEQ ID NO: 13, SEQ ID NO: 14 and SEQ ID NO: 15. Each possibility represents a separate embodiment of the present invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description

### BRIEF DESCRIPTION OF THE FIGURES

FIG. 1A-F:

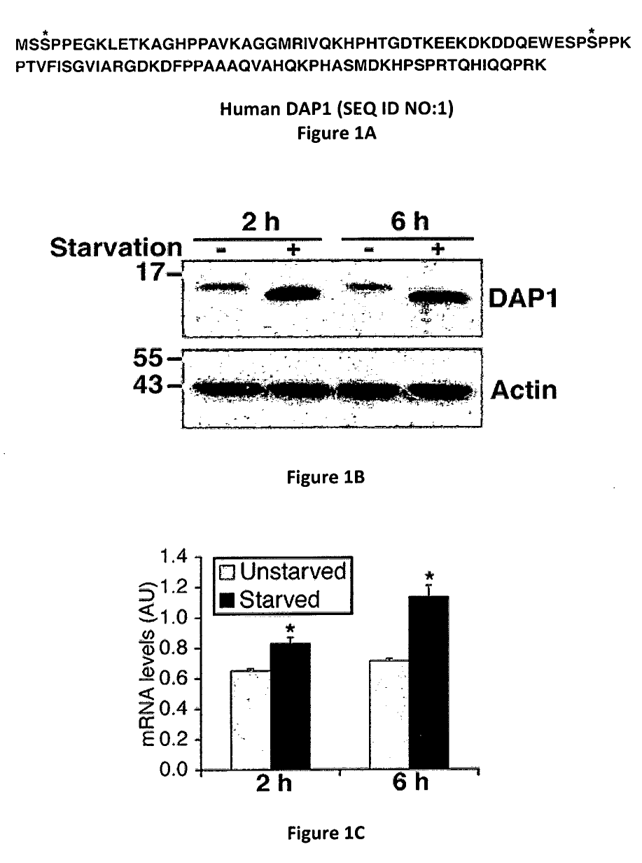

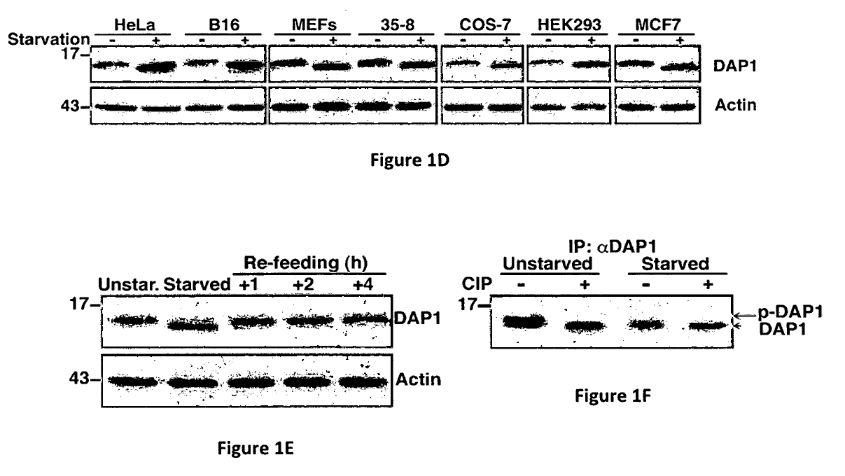

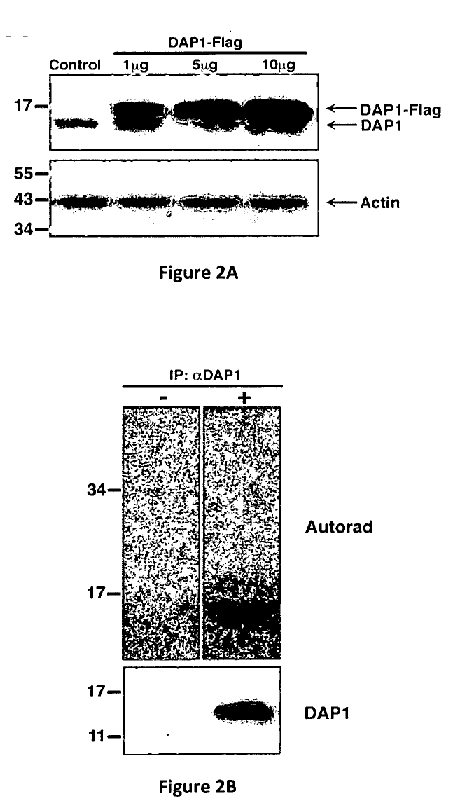

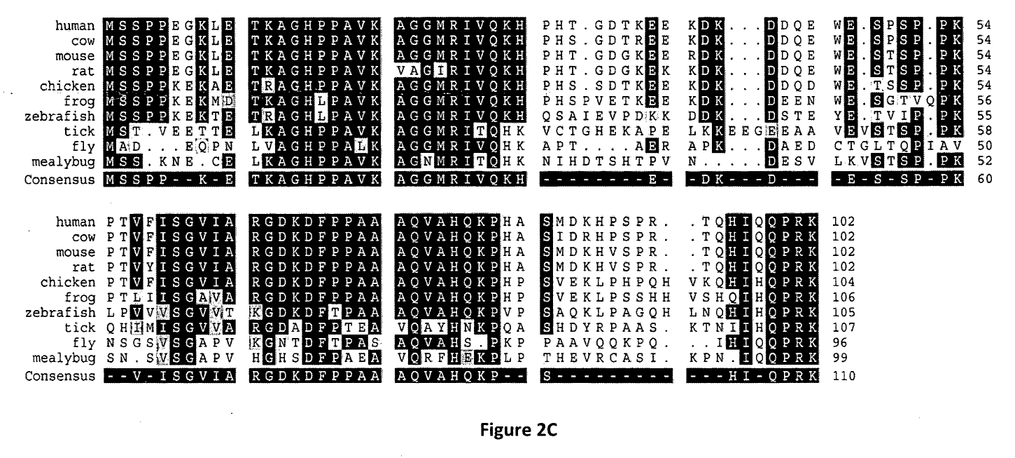

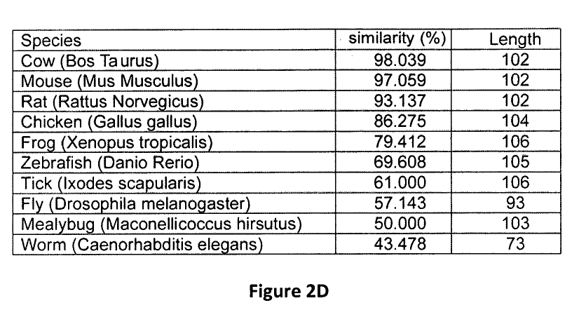

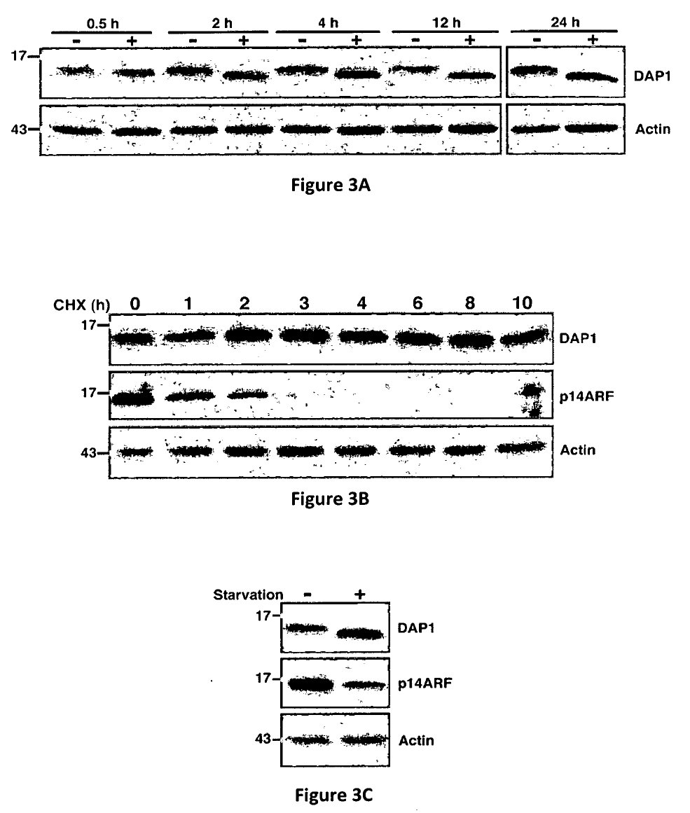

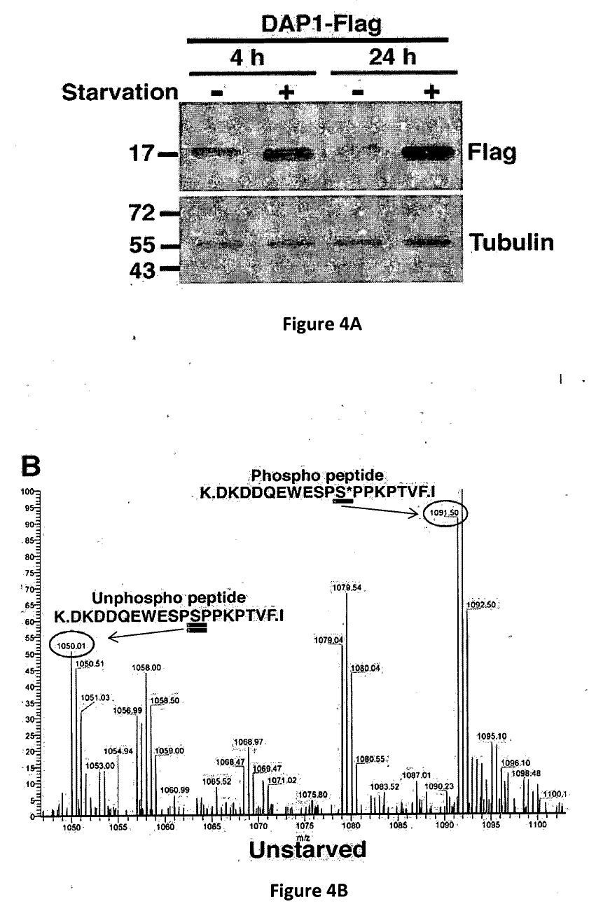

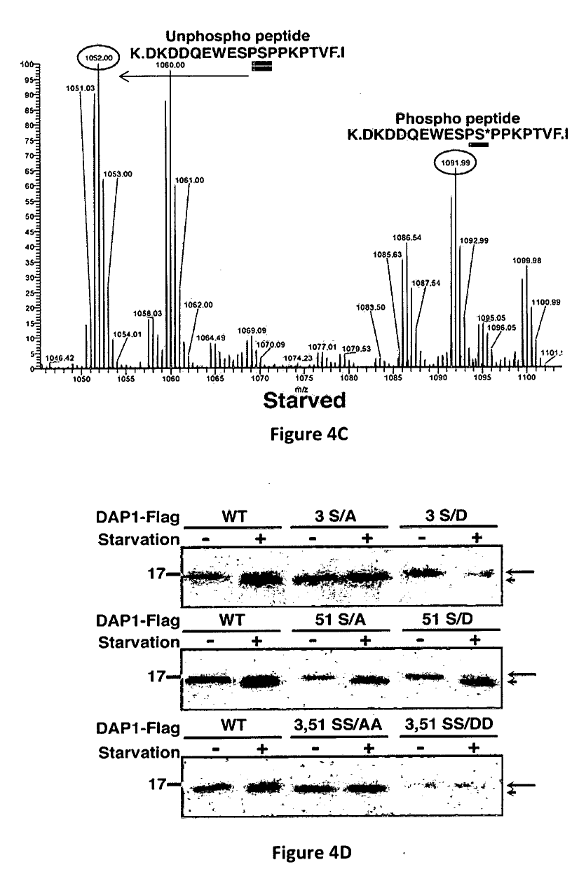

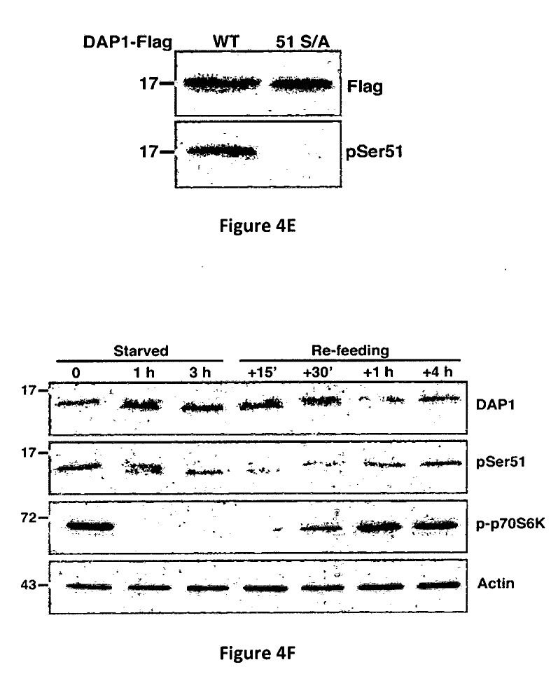

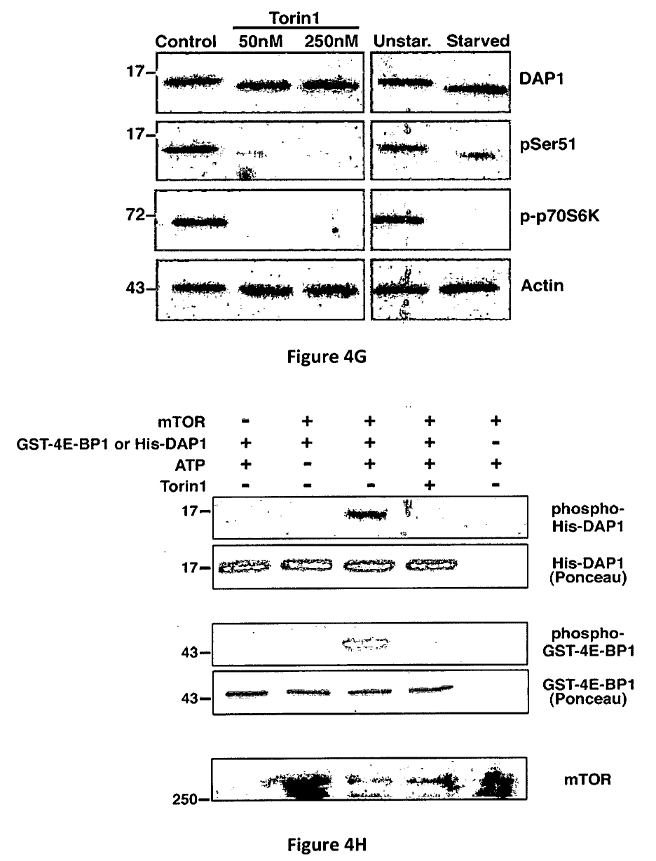

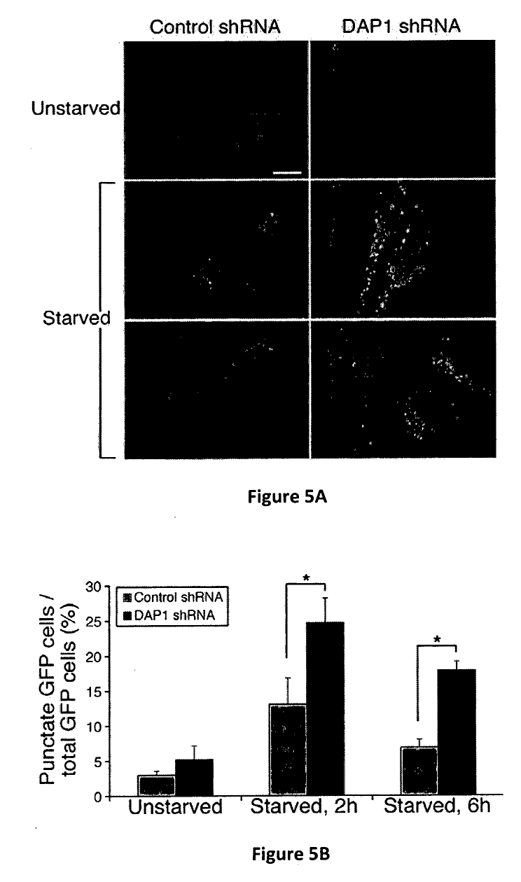

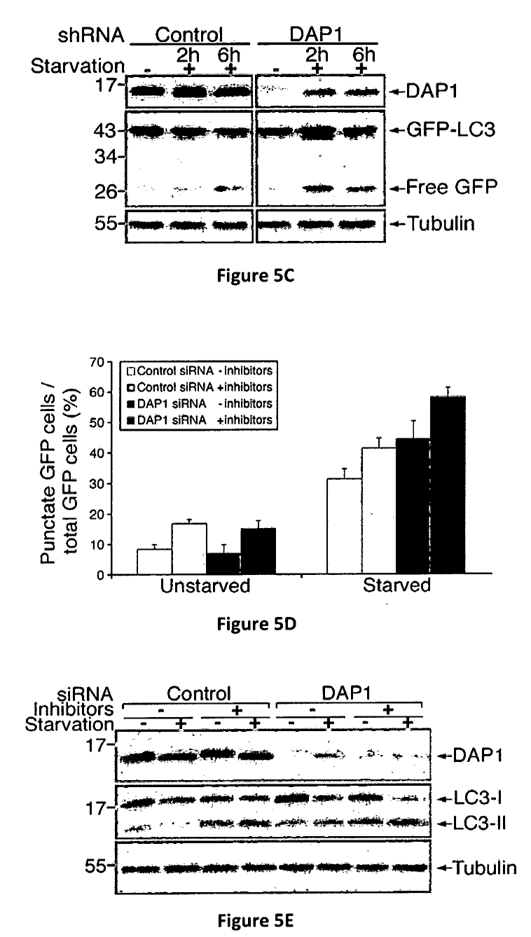

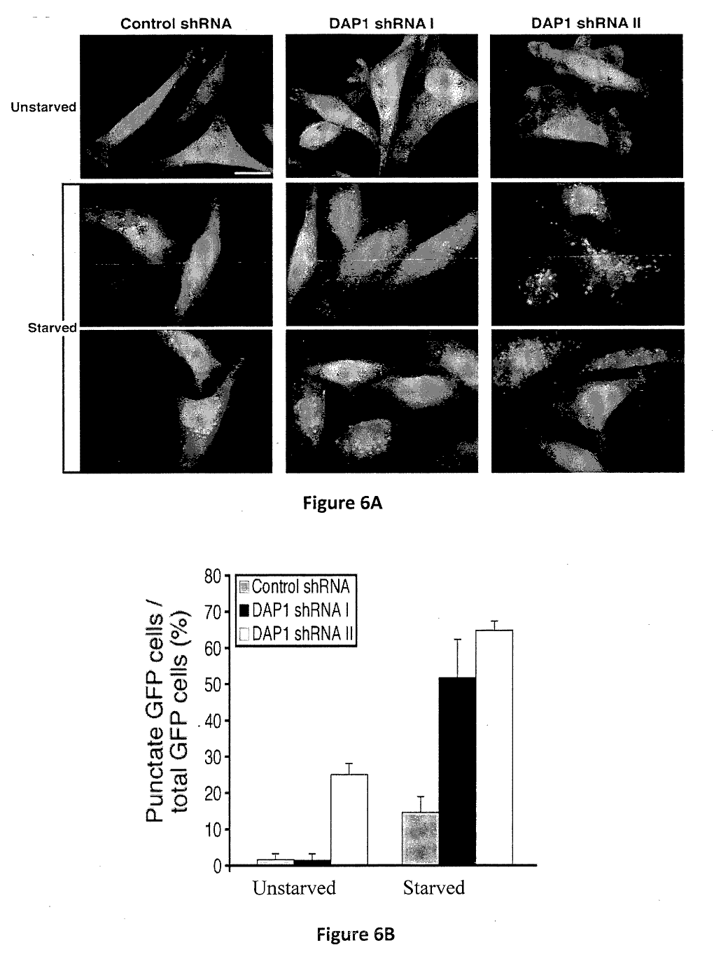

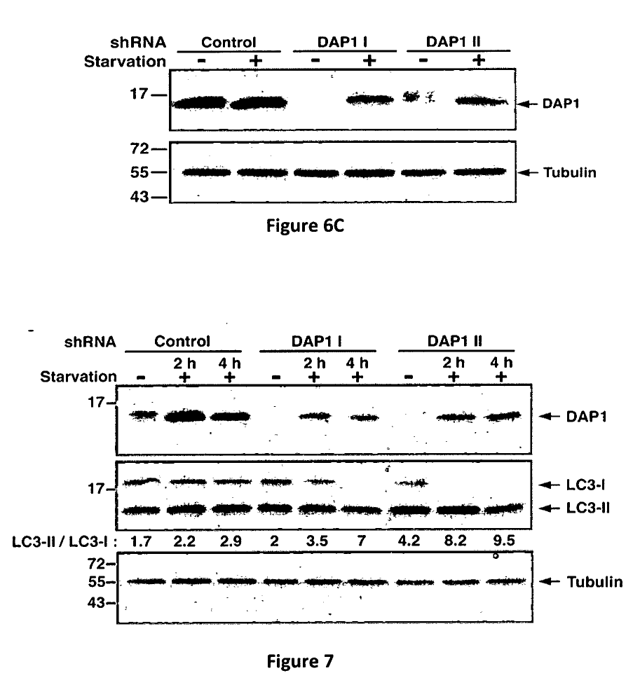

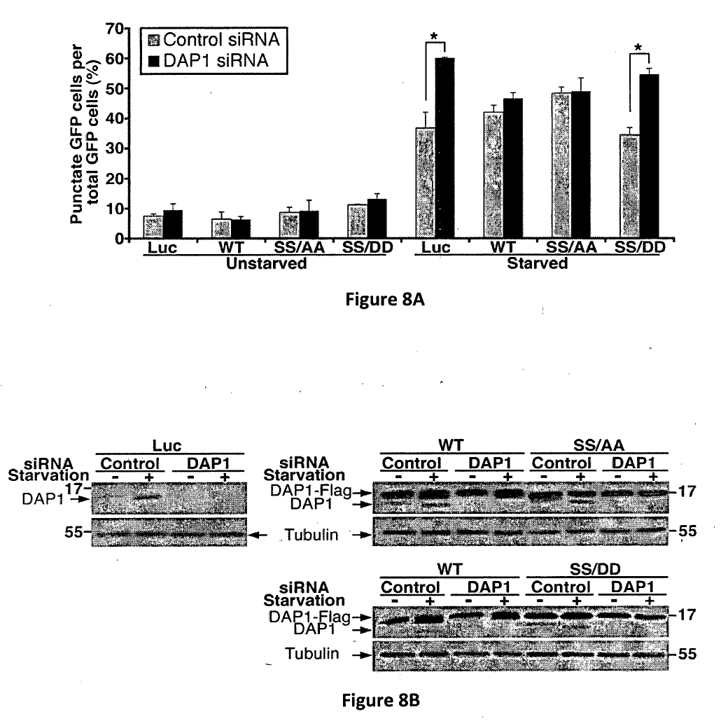

DAP1 is regulated at the expression and phosphorylation levels during nutrient starvation (FIG. 1A) Human DAP1 amino acid sequence (SEQ ID NO: 1). Proline residues are marked in gray. Serines 3 and 51 are marked with asterisks. (FIGS. 1B and 1C) DAP1 protein levels and the corresponding mRNA steady state levels measured by western blot analysis (B) and Real-Time PCR(C), following amino acid starvation for 2 or 6 h. Data presented are the mean±SD calculated from triplicates points. The asterisk denotes significance level of p<0.002, Student t-test. (FIG. 1D) DAP1 protein expression prior to or following starvation in different cell lines. (FIG. 1E) DAP1 protein expression in cells that were starved for 4 h and then supplemented with rich medium for up to 4 h (re-feeding). Actin was used as a loading control in FIGS. B-E. (FIG. 1F) Immunoprecipitated DAP1 was incubated in the presence or absence of CIP, prior to western blot analysis with anti-DAP1 antibody. Arrowhead points to a faster migrating band of DAP1; arrow points to the slower migrating band of p-DAP1.

FIG. 2 A-D:

DAP1 is a highly conserved phosphoprotein (FIG. 2A) Flag-tagged DAP1 was expressed in HeLa cells at different concentrations (from 1 to 10 μg plasmid per 9 cm plate). Immunoblotted cell lysates were reacted with anti-DAP1 antibodies. Endogenous DAP1 protein is 15 kDa in size (see control non transfected cells) while the exogenous overexpressed Flag-tagged DAP1 runs as a 17 kDa protein. Actin was used as a loading control. (FIG. 2B) HeLa cell cultures were labeled with ³³[P]-orthophoshphate and endogenous DAP1 was immunoprecipitated from the cell lysates (right lane); in the left panel equal amounts of cell extracts were similarly treated without adding the anti-DAP1 antibody. Upper panels: autoradiogram of immunoprecipitated DAP1; lower panel: western blot of the immunoprecipitated DAP1 using anti-DAP1 antibody. (FIG. 2C) Multiple alignments. The alignment was performed using ClustalW. The sequences used are: human—NP_{—}004385.1; cow—AAI03333.1; mouse—NP_{—}666169.1; rat—NP_{—}071971.1; chicken—CAG32591.1; frog—AAH96501.1; zebrafish—NP_{—}571647.1; tick—AAY66888.1; fly—NP_{—}610676.2; mealybug—ABM55613.1. The consensus sequence is displayed at the bottom. Black background indicates an identical residue to the consensus and gray background a similar residue. (FIG. 2D) Percent conservation across species. The sequences are the same as in FIG. 2C, with the addition of worm—NP_{—}492102.1.

FIG. 3 A-C:

DAP1 protein expression during starvation: (FIG. 3A) HeLa cells cultured in EBSS for 0.5 to 24 h. The cells (unstarved ‘−’; starved ‘+’) were harvested and DAP1 protein levels were analyzed by western blotting using anti-DAP1 antibodies. (FIG. 3B) DAP1 is a stable protein: DAP1 and p14ARF protein levels were analyzed by western blotting using anti-DAP1 and p14ARF antibodies following treatment with 20 μg/ml cycloheximide (CHX) (Sigma) for up to 10 hours. (FIG. 3C) Cells were either starved (+) or grown in rich medium (−) for 6 h and lysates were subjected to western blotting as in (B).

FIG. 4 A-H:

DAP1 is a direct substrate of mTOR (FIG. 4A) HEK293 polyclonal cells stably expressing DAP1-Flag were cultured in rich medium or were starved for 4 or 24 h and cell lysates were subjected to western blotting with anti-Flag antibodies. Tubulin was used as a loading control. (FIGS. 4B and C) The HEK293 polyclonal stable transfectants were either cultured in rich medium (FIG. 4B) or starved (FIG. 4C) for 4 h and DAP1-Flag was immunoprecipitated and resolved by SDS PAGE. The gel was stained with Gel-code and the DAP1-Flag bands in each fraction were excised and analyzed by LC-MS/MS as shown in FIG. 4B and FIG. 4C respectively. Each peak represents a peptide; unphosphorylated peptides containing Ser51 are doubly underlined), phosphopeptides containing phosphorylated Ser51 are underlined and marked with an asterisk). The graph height represents the relevant abundance of the peptide in the fraction. (FIG. 4D) HEK293T cells transfected with WT or mutant DAP1 at the indicated residue or residues were cultured in rich medium or starved for 6 h. Lysates were subjected to western blotting with anti-Flag antibodies. Arrowhead points to the faster migrating band; arrow points to the slower migrating band. (FIG. 4E) HeLa cells transfected with WT or Ser51 Ala mutant DAP1-Flag were cultured in rich medium or starved for 6 h. Lysates were subjected to western blotting with anti-Flag and anti-phospho-Ser51 DAP1 (pSer51) antibodies. (FIG. 4F) Lysates from HeLa cells that were starved for 3 h and then re-cultured in complete medium (Re-feeding) for up to 4 h were subjected to western blotting with anti-DAP1, anti-phospho-Ser51 DAP1 (pSer51) and anti-phospho-Thr389 p70S6K (p-p70S6K) antibodies. (FIG. 4G) HeLa cells were treated with Torin1 or DMSO (control) or cultured in rich medium (Unstarv.) or in EBSS (Starved) for 3 h. Lysates were subjected to western blotting as in FIG. 4F. (FIG. 4H) Immunopurified mTOR (lower panel) was incubated with recombinant His-DAP1 or GST-4E-BP1, in the presence or absence of ATP or Torin1. Equal quantities of substrate assayed was verified by Ponceau staining.

FIG. 5.

DAP1 knockdown increases autophagic flux. (FIG. 5A) A polyclonal population of HeLa cells stably expressing GFP-LC3 was transfected with DAP1-targeting shRNA or HcRed shRNA as control for 5 days and then starved for 2 or 6 h. Cells were fixed with 3.7% formaldehyde and were analyzed by fluorescent microscopy. Scale bar, 20 μm. (FIG. 5B) Quantitation of the percentage of cells with punctate GFP-LC3 fluorescence per total GFP-LC3-positive cells. Data represent mean±SD calculated from triplicates of 100 transfected cells each. *, p<0.02. (FIG. 5C) Western blot analysis with indicated antibodies of extracts from GFP-LC3 stably expressing cells transfected with DAP1 or HcRed shRNA. (FIG. 5D) HeLa cells stably expressing GFP-LC3 (clone 7) were transfected with DAP1 or HcRed siRNA for 3 days and then either cultured in rich medium or starved in the presence or absence of lysosomal inhibitors (E64d (10 μg/ml)+pepstatin A (10 μg/ml)) for 4 h. Cells were fixed as in FIG. 5A and analyzed as in FIG. 5B. (FIG. 5E) Western blot analysis of cell extracts prepared from siRNA-transfected cells (clone 7), reacted with the indicated antibodies.

FIG. 6.

Knocking down DAP1 enhances autophagosomes accumulation during starvation. (FIG. 6A) HeLa cells stably expressing GFP-LC3 (clone 7) were transfected with two different DAP1-targeting shRNAs or HcRed shRNA as control. After 5 days, the cells were cultured in DMEM with 10% FBS (Unstarved) or in EBSS (Starved) for 2 h. Cells were fixed with 3.7% formaldehyde and were analyzed by fluorescent microscopy. Scale bar, 20 μm. (FIG. 6B) Quantitation of the percentage of cells with punctate GFP-LC3 fluorescence per total GFP-LC3-positive cells. Data represent mean±SD calculated from triplicates of 100 transfected cells each. (FIG. 6C) Western blot analysis of extracts of shRNA transfected cells, reacted with anti-DAP1 or anti-Tubulin (loading control) antibodies.

FIG. 7.

DAP1 knockdown enhances LC3 lipidation during starvation. HeLa cells were transfected with 2 different DAP1-targeting shRNAs or with HcRed shRNA as control. After 5 days the cells were either starved or grown in rich medium for 2 or 4 h. Lysates were western blotted for DAP1, LC3 or tubulin, as a loading control. Densitometric quantitation of LC3-I and LC3-II after normalization to tubulin was conducted and the ratio between LC3-II to LC3-I is presented.

FIG. 8.

Dephosphorylation of DAP1 activates its suppressive function in autophagy. (FIG. 8A) Clone 7 GFP-LC3 HeLa cells were co-transfected with siRNA to endogenous DAP1 or HcRed, together with Luciferase, WT or mutated DAP1-Flag plasmids for 3 days and then either cultured in rich medium (Unstarved) or starved of amino acids for 4 h. Cells were fixed and analyzed by fluorescent microscopy. Graph indicates the percentage of cells with punctate GFP-LC3 fluorescence per total GFP-LC3-positive cells, as a mean±SD calculated from triplicates of 100 transfected cells each. *, p<0.03. (FIG. 8B) Western blot analysis of lysates from FIG. 8A reacted with anti-DAP1 or anti-Tubulin (as loading control) antibodies.

### DETAILED DESCRIPTION OF THE INVENTION

The present invention identifies the important role of DAP1 in autophagy regulation. This invention presents DAP1 as a suppressor of autophagy and as a novel substrate of mTOR. It is herein disclosed for the first time that DAP1's suppressive function is acquired once the inhibitory phosphorylations are removed by mTOR inactivation, and thus restricts the intensity of the autophagic flux to maintain the continuous benefits of the autophagic process under stress. Without wishing to be bound by any theory or mechanism of action, these findings fit a ‘Gas and Brake’ model in which mTOR, a regulator of autophagic induction, also simultaneously controls the activity of a specific balancing brake aimed at limiting the extent of the autophagic response to maintain the proper homeostatic balance.

“Autophagy” as used herein refers to a variety of tightly-regulated catabolic processes which involve the degradation of a cell's own components through the lysosomal machinery and play a normal part in cell growth, development, and homeostasis, helping to maintain a balance between the synthesis, degradation, and subsequent recycling of cellular products. The most well-known catabolic process of autophagy involves the formation of a membrane around a targeted region of the cell, separating the contents from the rest of the cytoplasm. The resultant vesicle then fuses with a lysosome and subsequently degrades the contents. While autophagy was originally viewed as an inducible cellular mechanism to provide an energy source during short-term starvation, it has subsequently been shown that constitutive autophagy mediates the elimination of protein aggregates or damaged organelles and thus plays a protective role in multiple cell types. On the other hand, high levels of autophagy can lead to cell death. These observations suggest that autophagic activity needs to be closely monitored and regulated within a cell. Consistent with this notion, de-regulation of autophagic function has been proposed to participate in neurodegenerative disease and cancer, the innate immune response, as well as aging.

The term “stress” as used herein refers to physiological or psychological perturbances which disrupt an individual's homeostatic balance, and can be as diverse as injury, starvation, temperature extremes metabolic disruption, oxygen radicals and infection with intracellular pathogens. Exposure to stress and in particular to prolonged stress, may lead to stress-related conditions including cancer, myopathy, steroid diabetes, hypertension, peptic ulcers, reproductive impairment, psychogenic dwarfism and immunosuppression. ‘Prolong stress’ also referred to as ‘chronic stress’ as used herein refers to stress that exists for weeks, months or even years (e.g. prolonged osteomyelitis infection and sepsis, chronic exposure to toxic chemicals, or chronic tobacco abuse).

The present invention provides compositions and methods for the modulation of autophagy by altering the phosphorylation of Death Associated Protein 1 (DAP1). The compositions and methods of the present invention are useful for suppressing autophagy in target cell populations. According to some embodiments, the compositions and methods of the present invention are useful for promoting autophagy in target cell populations. According to some embodiments the target cell is a cell under stress. According to some embodiments, the target cell is a cell under prolonged stress. According to another embodiment, the target cell is a nutrient deprived. According to other embodiments, the target cell is a cancer cell (e.g., a nutrient deprived cancer cell).

DAP1 Variants

DAP1 is a small (˜15 kDa), ubiquitously expressed protein, rich in prolines and lacking known functional motifs. The human DAP1 has the amino acid sequence as set forth in SEQ ID NO:1 (mssppegkle tkaghppavk aggmrivqkh phtgdtkeek dkddqewesp sppkptvfis gviargdkdf ppaaaqvahq kphasmdkhp sprtqhiqqp rk). It is to be appreciated that the present invention encompasses variants of other mammalian DAP1 such as mouse, bovine, pig, and the like.

The present invention identified for the first time DAP1 as a suppressor of autophagy and as a novel direct substrate of mTOR. As exemplified herein below, DAP1 is functionally silent in cells growing under rich nutrient supplies through mTOR-dependent inhibitory phosphorylation on two sites, which were mapped to Ser3 and Ser51 of human DAP1. It is further exemplified herein below, that during amino acid starvation, mTOR activity is turned off resulting in a rapid reduction in the phosphorylation of DAP1.

According to some embodiments, the present invention provides human DAP1 variants capable of regulating autophagy in a target cell. Typically, the target cell is a cell under stress as defined hereinabove. Some of the human DAP1 variants according to embodiments of the invention are capable of decreasing or suppressing autophagy; whereas other human DAP1 variants are capable of increasing or inducing autophagy.

The term “DAP1 variant” refers herein to a DAP1 protein comprising a modified or altered amino acid sequence compared to the naturally occurring DAP1 (SEQ ID NO:1) wherein at least one phosphorylation site selected from serine 3 and serine 51 is altered, wherein the variant modulates autophagy in a target cell. The term “altered phosphorylation site” as used herein refers to an alteration of a phosphorylation site by an amino acid substitution and/or by chemical modification. In some embodiments, the altered phosphorylation site relates to a serine residue substituted with a phospho-silencing residue. In other embodiments, the altered phosphorylation site relates to a serine residue substituted with a phospho-mimicking residue.

The term “phosphorylation” has the meaning known in the art, e.g., the term refers to a phosphate transfer in which a phosphate group from a donor molecule is transferred to an acceptor molecule. Specifically, the term refers to the chemical addition of a phosphate group (e.g., PO₄²—) to a DAP1. Under cellular conditions phosphorylation is achieved enzymatically by an enzyme such as a kinase. Typically, phosphorylation usually occurs on serine, threonine, and tyrosine residues in eukaryotic proteins. The present invention relates to the phosphorylation on serine residues, specifically of serine at position 3 and/or serine at position 51 of human DAP1 (SEQ ID NO:1).

In some embodiments, the methods of the invention comprise altering the phosphorylation of DAP1. The phrase “altering the phosphorylation of DAP1” includes enhancing DAP1 phosphorylation and reducing or inhibiting DAP1 phosphorylation. The term “reducing or inhibiting DAP1 phosphorylation” as used herein includes preventing phosphorylation of at least one phosphorylation site selected from Ser3 and Ser51 of human DAP1. This term also includes decreasing the extent of phosphorylation of DAP1 by preventing phosphorylation occurring at one or more phosphorylation sites, or as a result of dephosphorylation occurring at one or more phosphorylated sites on DAP1.

The term “dephosphorylation” has the meaning known in the art, e.g., the term refers to the chemical removal of a phosphate group (e.g., PO₄²—) from a biochemical entity such as a protein (e.g., DAP1). Under cellular conditions, dephosphorylation is achieved enzymatically by an enzyme such as a phosphatase.

The term “enhancing DAP1 phosphorylation” as used herein includes phosphorylation of DAP1 on specific residues, such as serine residues located at position 3 and/or serine at position 51 of human DAP1 (SEQ ID NO:1).

Techniques are well known in the art for analyzing phosphorylation modification states. For example, phosphorylation may be determined by the use of antibodies to phospho-epitopes to detect a phosphorylated polypeptide by Western analysis, for example, as described in the Examples herein.

Human DAP1 variants capable of suppressing autophagy are variants in which serine residue at position 3 and serine residue at position 51 of said human DAP1 variant are substituted with a phospho-silencing residue. Alternatively, only the serine residue at position 3 is substituted with a phospho-silencing residue; alternatively, only the serine residue at position 51 is substituted with a phospho-silencing residue. Each possibility represents a separate embodiment of the invention.

According to particular embodiments, said human DAP1 variant capable of suppressing autophagy (e.g., in a target cell) is selected from the amino acid sequence as set forth in SEQ ID NOs: 2-4, and SEQ ID NO: 13-15. In one embodiment, said human DAP1 variant consists of the amino acid sequence as set forth in SEQ ID NO: 2. In another embodiment, said human DAP1 variant consists of the amino acid sequence as set forth in SEQ ID NO: 3. In another embodiment, said human DAP1 variant consists of the amino acid sequence as set forth in SEQ ID NO: 4. In another embodiment, said human DAP1 variant consists of the amino acid sequence as set forth in SEQ ID NO: 13. In another embodiment, said human DAP1 variant consists of the amino acid sequence as set forth in SEQ ID NO: 14. In another embodiment, said human DAP1 variant consists of the amino acid sequence as set forth in SEQ ID NO: 15.

Human DAP1 variants capable of inducing autophagy are variants in which serine residue at position 3 and serine residue at position 51 of said human DAP1 variant are substituted with a phospho-mimicking residue. Alternatively, only the serine residue at position 3 is substituted with a phospho-mimicking residue; alternatively, only the serine residue at position 51 is substituted with a phospho-mimicking residue. Each possibility represents a separate embodiment of the invention.

According to particular embodiments, said human DAP1 variant capable of inducing autophagy (e.g., in a target cell) is selected from the amino acid sequence as set forth in SEQ ID NOs: 5-7. In one embodiment, said human DAP1 variant consists of the amino acid sequence as set forth in SEQ ID NO: 5. In another embodiment, said human DAP1 variant consists of the amino acid sequence as set forth in SEQ ID NO: 6. In another embodiment, said human DAP1 variant consists of the amino acid sequence as set forth in SEQ ID NO: 7.

A “phospho-silencing residue” as used herein refers to a residue which is incapable of phosphorylation (e.g., a nonphosphorylatable residue) and is other than a phospho-mimicking residue. According to one embodiment, the phospho-silencing residue is selected from the group consisting of alanine, isoleucine, leucine, asparagines, lysine, methionine, phenylalanine, glutamine, tryptophan, glycine, valine, proline, arginine and histidine. According to some embodiments, the phospho-silencing residue is alanine.

A “phospho-mimicking residue” as used herein refers to a residue which is not phosphorylated but displays physico-chemico properties similar to a residue carrying a phosphate ion (phosphorylated residue) such as for example aspartic acid or glutamic acid. According to one embodiment, the phospho-mimicking residue is negatively charged. According to another embodiment the phospho-mimicking residue is negatively charged at pH above the pI of the phospho-mimicking residue. According to another embodiment, the phospho-mimicking residue is negatively charged at physiological pH (pH=7.4).

According to another aspect, the present invention provides an isolated polynucleotide encoding the human DAP1 variants of the present invention, wherein serine at position 3, or serine at position 51 or both are substituted with a phospho-silencing residue. According to another embodiment the sequence of human DAP1 carrying phospho-silencing mutants at position serine 3 and at position serine 51 comprises the amino acid sequence msX¹ ppegkle tkaghppavk aggmrivqkh phtgdtkeek dkddqewesp X² ppkptvfis gviargdkdf ppaaaqvahq kphasmdkhp sprtqhiqqp rk; wherein X¹ and X² are each independently a phospho-silencing residue. In one embodiment, the human DAP1 variant has the amino acid sequence as set forth in SEQ ID NO:2.

According to another embodiment human DAP1 variant is a homolog of SEQ ID NO: 2, SEQ ID NO: 3 or SEQ ID NO: 4. In another embodiment, the human DAP1 variant sequence is a fragment of SEQ ID NO: 2, SEQ ID NO: 3 or SEQ ID NO: 4. In another embodiment, the human DAP1 variant sequence is a homolog of a fragment of SEQ ID NO: 2, SEQ ID NO: 3 or SEQ ID NO: 4. In different embodiments, “homolog” may refer e.g. to any degree of homology disclosed herein. Each possibility represents a separate embodiment of the present invention.

According to yet another aspect, the present invention provides an isolated polynucleotide encoding the human DAP1 variants of the present invention, wherein serine at position 3, or serine at position 51 or both are substituted with a phospho-mimicking residue. According to another embodiment the sequence of human DAP1 carrying phospho-mimicking mutants at position serine 3 and at position serine 51 comprises the amino acid sequence: msX³ ppegkle tkaghppavk aggmrivqkh phtgdtkeek dkddqewesp X⁴ ppkptvfis gviargdkdf ppaaaqvahq kphasmdkhp sprtqhiqqp rk; wherein X³ and X⁴ are each independently a phospho-mimicking residue. In one embodiment, the human DAP1 variant has the amino acid sequence as set forth in SEQ ID NO:5. According to another embodiment the sequence of human DAP1 carrying a phospho-mimicking mutant at position serine 3 comprises the amino acid sequence as set forth in SEQ ID NO:6. According to another embodiment the sequence of human DAP1 carrying a phospho-mimicking mutant at position serine 51 comprises the amino acid sequence as set forth in SEQ ID NO:7. According to another embodiment human DAP1 variant is a homolog of SEQ ID NO: 5, SEQ ID NO: 6 or SEQ ID NO: 7. In another embodiment, the human DAP1 variant sequence is a fragment of SEQ ID NO:5, SEQ ID NO: 6 or SEQ ID NO: 7. In another embodiment, the human DAP1 variant sequence is a homolog of a fragment of SEQ ID NO: 5, SEQ ID NO: 6 or SEQ ID NO: 7. Each possibility represents a separate embodiment of the present invention.

As used herein, the term “variant” refers to substantially similar sequences possessing common qualitative biological activities. A polypeptide or polynucleotide variant includes a homolog, analog, extension or fragment of a polypeptide or polynucleotide sequence useful for the invention. In specific embodiments a DAP1 variant of the invention refers to a DAP1 polypeptide, or polynucleotide encoding same, comprising a modified or altered amino acid sequence compared to the naturally occurring DAP1 (SEQ ID NO:1) wherein at least one phosphorylation site selected from serine 3 and serine 51 is altered, and wherein the variant modulates autophagy in a target cell

The term “polypeptide” as used herein refers to a linear series of natural, non-natural and/or chemically modified amino acid residues connected one to the other by peptide bonds. The amino acid residues are represented throughout the specification and claims by either one or three-letter codes, as is commonly known in the art.

With respect to peptides, the term “analog” refers to a polypeptide comprising at least one altered amino acid residue by an amino acid substitution, addition, deletion, or chemical modification, as compared with the native polypeptide. Polypeptide analogs include amino acid substitutions and/or additions with naturally occurring amino acid residues, and chemical modifications such as, for example, enzymatic modifications, typically present in nature. Polypeptide analogs also include amino acid substitutions and/or additions with non-natural amino acid residues, and chemical modifications which do not occur in nature.

In general, analogs typically will share at least 50% amino acid identity to the native sequences disclosed in the present invention, in some instances the analogs will share at least 60% amino acid identity, at least 70%, 80%, 90%, and in still other instances the analogs will share at least 95% amino acid identity to the native polypeptides.

With respect to polynucleotide, encompassed within the term “variant” are chemically modified natural and synthetic nucleotide molecules (derivatives). Also encompassed within the term “variant” are substitutions (conservative or non-conservative), additions or deletions within the nucleotide sequence of the molecule, as long as the required function is sufficiently maintained. Polynucleotides variants may share at least 50%, 55%, 60%, 65%, 70%, 75%, 80%, 85%, 90%, 95%, 96%, 97%, 98% or 99% sequence identity (homology). In different embodiments, “homolog” may refer e.g. to any degree of homology disclosed herein.

Various methods of introducing isolated polynucleotide into cells exist and are well-known in the art. In one example, one can introduce the isolated polynucleotide by (a) recovering cells (e.g., stressed cells) from a subject, (b) introducing the isolated polynucleotide encoding a human DAP1 variant (e.g., SEQ ID NOs: 2-4) into the cells; and (c) reintroducing the cells of step (b) into the subject so as to treat the subject.

The term “polynucleotide” as used herein refers to an oligonucleotide, polynucleotide or nucleotide and fragments or portions thereof, and to DNA or RNA of genomic or synthetic origin, which may be single- or double-stranded, and represent the sense or antisense strand. The term should also be understood to include, as equivalents, analogs of either RNA or DNA made from nucleotide analogs, and, as applicable to the embodiment being described.

An “isolated polynucleotide” refers to a polynucleotide segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to polynucleotides, which have been substantially purified from other components, which naturally accompany the polynucleotide in the cell, e.g., RNA or DNA or proteins. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA, which is part of a hybrid gene encoding additional polypeptide sequence, and RNA such as mRNA.

The term “encoding” refers to the inherent property of specific sequences of nucleotides in an isolated polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a peptide or protein if transcription and translation of mRNA corresponding to that gene produces the peptide or protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the peptide or protein or other product of that gene or cDNA

The invention also includes degenerate nucleic acids which include alternative codons to those present in the native materials. For example, leucine residues are encoded by the codons TTA, TTG, CTT, CTC, CTA and CTG. Each of the six codons is equivalent for the purposes of encoding a leucine residue. Thus, it will be apparent to one of ordinary skill in the art that any of the leucine-encoding nucleotide triplets may be employed to direct the protein synthesis apparatus, in vitro or in vivo, to incorporate a leucine residue into elongating polypeptides of the invention. Similarly, nucleotide sequence triplets which encode other amino acid residues include, but are not limited to: CCA, CCC, CCG and CCT (proline codons); CGA, CGC, CGG, CGT, AGA and AGG (arginine codons); ACA, ACC, ACG and ACT (threonine codons); AAC and AAT (asparagine codons); and ATA, ATC and ATT (isoleucine codons). Other amino acid residues may be encoded similarly by multiple nucleotide sequences. Thus, the invention embraces degenerate nucleic acids that differ from the biologically isolated nucleic acids in codon sequence due to the degeneracy of the genetic code.

A polynucleotide of the present invention can be produced using recombinant DNA technology (e.g., polymerase chain reaction (PCR) amplification, cloning) or chemical synthesis. Nucleic acid sequences include natural nucleic acid sequences and homologs thereof, comprising, but not limited to, natural allelic variants and modified nucleic acid sequences in which nucleotides have been inserted, deleted, substituted, and/or inverted in such a manner that such modifications do not substantially interfere with the nucleic acid molecule's ability to encode the recombinant polypeptides of the present invention.

As used herein, the term “construct” means any recombinant polynucleotide molecule such as a plasmid, cosmid, virus, autonomously replicating polynucleotide molecule, phage, or linear or circular single-stranded or double-stranded DNA or RNA polynucleotide molecule, derived from any source, capable of genomic integration or autonomous replication, comprising a polynucleotide molecule where one or more polynucleotide molecule has been linked in a functionally operative manner, i.e. operably linked. A recombinant construct will typically comprise the polynucleotides of the present invention operably linked to transcriptional initiation regulatory sequences, such as to direct the transcription of the polynucleotide in the intended host cell. Both heterologous and non-heterologous (i.e., endogenous) promoters can be employed to direct expression of the nucleic acids of the present invention.

As used herein, the term “vector” refers to any recombinant polynucleotide construct that may be used for the purpose of transformation, i.e. the introduction of heterologous DNA into a host cell. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”.

An “expression vector” as used herein refers to a nucleic acid molecule capable of replication and expressing a gene of interest when transformed, transfected or transduced into a host cell. The expression vectors comprise one or more phenotypic selectable markers and an origin of replication to ensure maintenance of the vector and to, if desired, provide amplification within the host. Selectable markers include, for example, sequences conferring antibiotic resistance markers, which may be used to obtain successful transformants by selection, such as ampicillin, tetracycline and kanamycin resistance sequences, or supply critical nutrients not available from complex media. The expression vector further comprises a promoter. In the context of the present invention, the promoter must be able to drive the expression of the polypeptide within the cells. Many viral promoters are appropriate for use in such an expression vector (e.g., retroviral ITRs, LTRs, immediate early viral promoters (IEp) (such as herpes virus IEp (e.g., ICP4-IEp and ICP0-IEp) and cytomegalovirus (CMV) IEp), and other viral promoters (e.g., late viral promoters, latency-active promoters (LAPs), Rous Sarcoma Virus (RSV) promoters, and Murine Leukemia Virus (MLV) promoters). Other suitable promoters are eukaryotic promoters, which contain enhancer sequences (e.g., the rabbit β-globin regulatory elements), constitutively active promoters (e.g., the β-actin promoter, etc.), signal and/or tissue specific promoters (e.g., inducible and/or repressible promoters, such as a promoter responsive to TNF or RU486, the metallothionine promoter, etc.), and tumor-specific promoters. Suitable expression vectors may be plasmids derived, for example, from pBR322 or various pUC plasmids, which are commercially available. Other expression vectors may be derived from bacteriophage, phagemid, or cosmid expression vectors, all of which are described in sections 1.12-1.20 of Sambrook et al., (Molecular Cloning: A Laboratory Manual. 3^{rd} edn., 2001, Cold Spring Harbor Laboratory Press). Isolated plasmids and DNA fragments are cleaved, tailored, and ligated together in a specific order to generate the desired vectors, as is well known in the art (see, for example, Sambrook et al., ibid).

Methods for manipulating a vector comprising an isolated polynucleotide are well known in the art (e.g., Sambrook et al., 1989, Molecular Cloning: A Laboratory Manual, 2d edition, Cold Spring Harbor Press, the contents of which are hereby incorporated by reference in their entirety) and include direct cloning, site specific recombination using recombinases, homologous recombination, and other suitable methods of constructing a recombinant vector. In this manner, an expression vector can be constructed such that it can be replicated in any desired cell, expressed in any desired cell, and can even become integrated into the genome of any desired cell.

The expression vector comprising the polynucleotide of interest is introduced into the cells by any means appropriate for the transfer of DNA into cells. Many such methods are well known in the art (e.g., Sambrook et al., supra; see also Watson et al., 1992, Recombinant DNA, Chapter 12, 2d edition, Scientific American Books, the contents of which are hereby incorporated by reference in their entirety). Thus, in the case of prokaryotic cells, vector introduction can be accomplished, for example, by electroporation, transformation, transduction, conjugation, or mobilization. For eukaryotic cells, vectors can be introduced through the use of, for example, electroporation, transfection, infection, DNA coated microprojectiles, or protoplast fusion. Examples of eukaryotic cells into which the expression vector can be introduced include, but are not limited to, ovum, stem cells, blastocytes, and the like.

According to another embodiment, the present invention provides a recombinant polynucleotide construct wherein a polynucleotide encoding any of the human DAP1 variants of the present invention, is operably linked to a transcription regulating sequences that will direct the transcription of the polynucleotide in the intended host cell. In another embodiment, the transcriptional regulating sequences are transcriptional initiation regulating sequences. The invention further provides vectors comprising the recombinant polynucleotide constructs encoding the human DAP1 variants of the invention, the vector being a plasmid or a virus. Consequently, the recombinant polynucleotide construct may be expressed in a host cell selected from eukaryotic and prokaryotic.

Pharmaceutical Compositions

According to another embodiment, the present invention provides a pharmaceutical composition comprising a therapeutically effective amount of an active agent selected from the group consisting of: (a) an isolated polypeptide comprising the amino acid sequence of a human DAP1 variant; (b) an isolated polynucleotide encoding the human DAP1 variant of (a); (c) an expression vector comprising the isolated polynucleotide of (b); and (d) a host cell transfected with the expression vector of (c); further comprising a pharmaceutically acceptable carrier, wherein said human DAP1 variant comprises at least one serine residue selected from serine 3 and serine 51 of human DAP1 (SEQ ID NO: 1) substituted with a phospho-silencing residue.

According to another embodiment, the present invention provides a pharmaceutical composition comprising a therapeutically effective amount of an active agent selected from the group consisting of: (a) an isolated polypeptide comprising the amino acid sequence of a human DAP1 variant; (b) an isolated polynucleotide encoding the human DAP1 variant of (a); (c) an expression vector comprising the isolated polynucleotide of (b); and (d) a host cell transfected with the expression vector of (c); further comprising a pharmaceutically acceptable carrier, wherein said human DAP1 variant comprises at least one serine residue selected from serine 3 and serine 51 of human DAP1 (SEQ ID NO: 1) substituted with a phospho-mimicking residue.

According to some embodiments, the present invention provides a pharmaceutical composition comprising as an active ingredient a recombinant polynucleotide construct comprising the isolated polynucleotide encoding a human DAP1 variant of the invention and a pharmaceutically acceptable carrier, excipient or diluent.

As used herein, a “pharmaceutical composition” refers to a preparation of one or more of the active ingredients described herein, e.g. a construct encoding a human DAP1 variant, wherein serine at position 3 and serine at position 51 of the human DAP1 variant is substituted with a phospho-silencing residue, with other components such as physiologically suitable carriers and excipients, or a construct encoding a human DAP1 variant, wherein serine at position 3 and serine at position 51 of the human DAP1 variant is substituted with a phospho-mimicking residue, with other components such as physiologically suitable carriers and excipients. The purpose of a pharmaceutical composition is to facilitate administration of a compound to a subject.

Hereinafter, the phrases “therapeutically acceptable carrier” and “pharmaceutically acceptable carrier”, which may be used interchangeably, refer to a carrier or a diluent that does not cause significant irritation to an organism and does not abrogate the biological activity and properties of the administered compound.

Herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils, and polyethylene glycols.

In another embodiment of the present invention, a therapeutic composition further comprises a pharmaceutically acceptable carrier. As used herein, a “carrier” refers to any substance suitable as a vehicle for delivering a polynucleotide molecule of the present invention to a suitable in vivo or in vitro site. As such, carriers can act as a pharmaceutically acceptable excipient of a therapeutic composition containing a polynucleotide molecule of the present invention. Preferred carriers are capable of maintaining a polynucleotide molecule of the present invention in a form that, upon arrival of the polynucleotide molecule to a cell, the polynucleotide molecule is capable of entering the cell and being expressed by the cell. Carriers of the present invention include: (1) excipients or formularies that transport, but do not specifically target a nucleic acid molecule to a cell (referred to herein as non-targeting carriers); and (2) excipients or formularies that deliver a nucleic acid molecule to a specific site in a subject or a specific cell (i.e., targeting carriers). Examples of non-targeting carriers include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity.

Therapeutic compositions of the present invention can be sterilized by conventional methods.

Targeting carriers are herein referred to as “delivery vehicles”. Delivery vehicles of the present invention are capable of delivering a therapeutic composition of the present invention to a target site in a subject. A “target site” refers to a site in a subject to which one desires to deliver a therapeutic composition. Examples of delivery vehicles include, but are not limited to, artificial and natural lipid-containing delivery vehicles. Natural lipid-containing delivery vehicles include cells and cellular membranes. Artificial lipid-containing delivery vehicles include liposomes and micelles. A delivery vehicle of the present invention can be modified to target to a particular site in a subject, thereby targeting and making use of a nucleic acid molecule of the present invention at that site. Suitable modifications include manipulating the chemical formula of the lipid portion of the delivery vehicle and/or introducing into the vehicle a compound capable of specifically targeting a delivery vehicle to a preferred site, for example, a preferred cell type. Specifically targeting refers to causing a delivery vehicle to bind to a particular cell by the interaction of the compound in the vehicle to a molecule on the surface of the cell. Suitable targeting compounds include ligands capable of selectively (i.e., specifically) binding another molecule at a particular site. Examples of such ligands include antibodies, antigens, receptors and receptor ligands. For example, an antibody specific for an antigen found on the surface of a target cell can be introduced to the outer surface of a liposome delivery vehicle so as to target the delivery vehicle to the target cell. Manipulating the chemical formula of the lipid portion of the delivery vehicle can modulate the extracellular or intracellular targeting of the delivery vehicle. For example, a chemical can be added to the lipid formula of a liposome that alters the charge of the lipid bilayer of the liposome so that the liposome fuses with particular cells having particular charge characteristics.

Preferably the pharmaceutical composition can also include a transfection agent such as DOTMA, DOPE, and DC-Chol (Tonkinson et al., 1996).

Another delivery vehicle comprises a recombinant virus particle. A recombinant virus particle of the present invention includes a therapeutic composition of the present invention, in which the recombinant molecules contained in the composition are packaged in a viral coat that allows entrance of DNA into a cell so that the DNA is expressed in the cell. A number of recombinant virus particles can be used, including, but not limited to, those based on adenoviruses, adeno-associated viruses, herpesviruses, lentivirus and retroviruses.

Other agents can be used are e.g. cationic lipids, polylysine, and dendrimers. Alternatively, naked DNA can be administered.

Therapeutic Use

According to some embodiments, the constructs, vectors and composition according to embodiments of the invention are useful in regulating autophagy, e.g., in a cell under stress.

According to some embodiments, the constructs, vectors and compositions of the invention are useful for the treatment of cancer, neurodegenerative diseases and other conditions which are the result of the exposure of a subject to stress and in particular to prolonged or chronic stress.

In some embodiments, the constructs, vectors and composition according to embodiments of the invention are useful in regulating autophagy in a nutrient deprived cancer cells. As used herein, “nutrient deprived cancer cells” may be cancer cells that are undergoing autophagy, wherein autophagy may occur due to a metabolic stress such as nutrient deprivation or hypoxia. Nutrient deprivation may be induced by a metabolic stress promoting agent. Cancer cells may be nutrient deprived due to a lack of blood flow or access to sufficient nutrients, wherein nutrient deprived cells may be deficient in oxygen, serum, amino acids, sugar (for example, glucose) or any combination thereof.

As used herein, the term “treatment” refers both to the treatment and to the prevention or prophylactic therapy.

The term “autophagy-associated disease or disorder” as used herein means a disorder that is caused by associated with, the result of, or otherwise related to aberrant autophagy and this term includes, but is not limited to, cancers, neurodegenerative disorders, and myopathies.

The term “cancer” as used herein means solid mammalian tumors as well as hematological malignancies. “Solid mammalian tumors” include cancers of the head and neck, lung, mesothelioma, mediastinum, esophagus, stomach, pancreas, hepatobiliary system, small intestine, colon, colorectal, rectum, anus, kidney, urethra, bladder, prostate, urethra, penis, testis, gynecological organs, ovaries, breast, endocrine system, skin central nervous system; sarcomas of the soft tissue and bone; and melanoma of cutaneous and intraocular origin. The term “hematological malignancies” includes childhood leukemia and lymphomas, Hodgkin's disease, lymphomas of lymphocytic and cutaneous origin, acute and chronic leukemia, plasma cell neoplasm and cancers associated with AIDS. In addition, a cancer at any stage of progression can be treated, such as primary, metastatic, and recurrent cancers. Information regarding numerous types of cancer can be found, e.g., from the American Cancer Society, or from, e.g., Wilson et al. (1991) Harrison's Principles of Internal Medicine, 12th Edition, McGraw-Hill, Inc. Both human and veterinary uses are contemplated.

The term “neurodegenerative disorders” as used herein means, but is not limited to, Huntington's disease, Parkinson's Disease, Alzheimer's Disease, dystonia, dementia, multiple sclerosis, Amyotrophic Lateral Sclerosis (ALS), and Creutzfeld-Jacob Disease.

The term “modulation” (and other formulations of this term, e.g., “modulate”, “modulates”, and “modulating”) or “regulation” when used herein in reference to a functional property or biological activity or process (e.g., autophagy), means the capacity to control or influence directly or indirectly, and by way of non-limiting examples, can alternatively mean inhibit or stimulate, hinder or promote, activate or suppress, and strengthen or weaken, or otherwise change a quality of such property, activity or process. In some embodiments, the modulation is manifested by an increase or a decrease in the expression level of a gene or protein, or the level of a functional property or biological activity from a cell, group of cells, subject, or subjects in which an agent has been administered as compared to controls in which the agent has not been administered. The modulation described herein can be determined by any appropriate assay, such as those described herein below. In certain instances, the level of a functional property or biological activity from the cell or group of cells is increased or decreased by at least about 5%, 10%, 20%, 25%, 35%, or 50% by administration of the agent as compared to control. In some embodiments, the level of a functional property or biological activity from the cell or group of cells is increased or decreased by at least about 60%, 70%, or 80% by administration of the agent as compared to control. In some embodiments, the level of a functional property or biological activity from the cell or group of cells is increased or decreased by at least about 85%, 90%, 95%, or 99% by administration of the agent as compared to control. Each possibility represents a separate embodiment of the invention.

Some embodiments of the present invention are directed to the use of a recombinant construct that expresses in cells of a subject, a human DAP1 variant wherein serine at position 3 and serine at position 51 of the human DAP1 variant are substituted with phospho-silencing residues, for the preparation of a medicament. In certain embodiments, the medicament is useful for treating or preventing a disorder associated with increased or abonormal autophagic flux, for treating or preventing cancer, for inhibiting tumor progression or metastasis, for inducing tumor regression, preventing neurodegenerative diseases and/or inhibiting the progression of a degenerative disease.

According to another embodiment, the present invention provides a method for treating cancer or inhibiting tumor progression in a subject in need thereof comprising expressing in cells of the subject a human DAP1 variant of the invention thereby treating cancer in the subject and/or inhibiting tumor progression in a subject. According to one embodiment the expression of the human DAP1 variant in cells of the subject suppresses the autophagic flux (e.g. abnormal autophagic flux), thereby treating cancer or inhibiting tumor progression in the subject. According to another embodiment, the expression of the human DAP1 variant in cells of the subject induces cell death, thereby treating cancer or inhibiting tumor progression in the subject. According to another embodiment, the expression of the human DAP1 variant in cells of the subject restores cell growth control, thereby treating cancer or inhibiting tumor progression in the subject.

According to another embodiment, the method for treating cancer of inhibiting tumor progression comprises the administration of a therapeutically effective amount of a recombinant polynucleotide construct comprising the isolated polynucleotide encoding the human DAP1 variant of the present invention. According to another embodiment, the recombinant polynucleotide construct is administered into the subject's cells ex vivo.

According to another embodiment, the subject to be treated by methods and composition of the present invention is selected from the group consisting of a subject displaying pathology resulting from cancer, a subject suspected of displaying pathology resulting from cancer, and a subject at risk of displaying pathology resulting from cancer.

In one embodiment, the method comprises administering to said subject a recombinant construct comprising at least one polynucleotide sequence encoding a human DAP1 variant wherein serine at position 3 and serine at position 51 of the human DAP1 variant are substituted with a phospho-silencing residue, the nucleic acid sequence being operably linked to at least one transcription-regulating sequence.

Another aspect of the present invention is directed to a method for treating cancer in a subject in need thereof, comprising administering to the subject a therapeutically effective amount of a recombinant construct comprising at least one polynucleotide sequence encoding a human DAP1 variant wherein seine at position 3 and/or serine at position 51 of the human DAP1 variant are substituted with phospho-silencing residues, the polynucleotide sequence being operably linked to at least one transcriptional initiation regulatory sequence.

In another aspect, the invention provides a method for inhibiting tumor progression in a subject in need thereof, comprising administering to the subject a therapeutically effective amount of a recombinant construct comprising at least one polynucleotide sequence encoding a human DAP1 variant wherein serine at position 3 and/or serine at position 51 of the human DAP1 variant are substituted with phospho-silencing residues, the polynucleotide sequence being operably linked to at least one transcriptional initiation regulatory sequence.

In another aspect, there is provided a method for inducing tumor regression in a subject in need thereof, comprising administering to the subject a therapeutically effective amount of a recombinant construct comprising at least one nucleic acid sequence encoding a human DAP1 variant wherein serine at position 3 and/or serine at position 51 of the human DAP1 variant are substituted with phospho-silencing residues, the polynucleotide sequence being operably linked to at least one transcriptional initiation regulatory sequence.

In another embodiment, tumors that may be treated according to the method of the present invention are those characterized by de-regulation of autophagy or de-regulation of autophagic function, this may be the result of reduced or no expression of DAP1 in at least a portion of the cells of the tumor and/or reduced or over expression or activity of the Ser/Thr mammalian target of rapamycin (mTOR) in at least a portion of the cells of the tumor. In another embodiment, the tumor is a solid tumor. For example, in some embodiments, the tumor may include pediatric solid tumors (e.g. Wilms' tumor, hepatoblastoma and embryonal rhabdomyosarcoma), wherein each possibility represents a separate embodiment of the present invention. In other embodiments, the tumor includes, but is not limited to, germ cell tumors and trophoblastic tumors (e.g. testicular germ cell tumors, immature teratoma of the ovary, sacrococcygeal tumors, choriocarcinoma and placental site trophoblastic tumors), wherein each possibility represents a separate embodiment of the present invention. According to additional embodiments, the tumor includes, but is not limited to, epithelial adult tumors (e.g. bladder carcinoma, hepatocellular carcinoma, ovarian carcinoma, cervical carcinoma, lung carcinoma, breast carcinoma, squamous cell carcinoma in head and neck, colon carcinoma, renal cell carcinoma and esophageal carcinoma), wherein each possibility represents a separate embodiment of the present invention. In yet further embodiments, the tumor includes, but is not limited to, neurogenic tumors (e.g. astrocytoma, ganglioblastoma and neuroblastoma), wherein each possibility represents a separate embodiment of the present invention. In another embodiment, the tumor is prostate cancer. In another embodiment, the tumor is pancreatic cancer. In other embodiments, the tumor includes, for example, Ewing sarcoma, congenital mesoblastic nephroma, gastric adenocarcinoma, parotid gland adenoid cystic carcinoma, duodenal adenocarcinoma, T-cell leukemia and lymphoma, nasopharyngeal angiofibroma, melanoma, osteosarcoma, uterus cancer and non-small cell lung carcinoma, wherein each possibility represents a separate embodiment of the present invention.

In certain embodiments, the pharmaceutical compositions of the present invention can be used to treat cancer alone or in combination with other established or experimental therapeutic regimens against cancer. Therapeutic methods for treatment of cancer suitable for combination with the present invention include, but are not limited to, chemotherapy, radiotherapy, phototherapy and photodynamic therapy, surgery, nutritional therapy, ablative therapy, combined radiotherapy and chemotherapy, brachiotherapy, proton beam therapy, immunotherapy, cellular therapy, and photon beam radiosurgical therapy.

Having now generally described the invention, the same will be more readily understood through reference to the following examples, which are provided by way of illustration and are not intended to be limiting of the present invention.

### EXAMPLES

### Materials and Methods

### DNA Constructs and Transfections

DAP1 cDNA was subcloned into pcDNA3, tagged at its C terminus with the Flag epitope. Serine (Ser) to alanine (Ala) or aspartic acid (Asp) mutations were generated by PCR-mediated site directed mutagenesis, using the QuickChange kit (STRATAGENE) according to the manufacturer's protocol. All mutations were confirmed by direct sequencing. The GFP-LC3 plasmid pEGFPC1-LC3 was a kind gift from N. Mizushima and T. Yoshimori. Control plasmid consisted of pcDNA3 expressing the luciferase gene. Cell lines were transfected by standard calcium phosphate technique. To generate stable cell lines expressing DAP1-Flag or GFP-LC3, HEK293 or HeLa cells were transfected with pcDNA3-DAP1-Flag or pEGFP-LC3, respectively and grown in the presence of G418 (1 mg/ml) (Calbiochem). Selected clones were individually isolated to create monoclonal populations, or resistant clones were pooled to generate a polyclonal population expressed average levels of the protein. For bacterial expression, DAP1 was subcloned into pET-15b containing N-terminal His tag. GST-4E-BP1 was a kind gift from N. Sonenberg. Both His-DAP1 and GST-4E-BP1 were expressed in BL21(DE3) (Novagen), and purifications were carried out using the standard GST method or HiTrap Nickel affinity column (GE Healthcare), respectively.

### RNA Interference

To generate pSUPER-based shRNA vectors targeting DAP1 and HcRed (control), 19-mer oligos corresponding to nucleotides 244-262 (DAP1 shRNA I; SEQ ID NO: 16) or 284-302 (DAP1 shRNA II; SEQ ID NO: 17) of human DAP1 (NM_{—}004394) and 99-117 of HcRed (AF363776) were annealed and ligated into the BglII and HindIII of the pSUPER vector (Brummelkamp T. R., et. Al., (2002), Science, 296:550-3). Alternatively, DAP1 was knocked down using siGENOME SMARTpool siRNA reagent (catalog# M-004415) or by individual ON-TARGETplus siRNA duplexes (catalog# J-004415-09) (Dharmacon). For control siRNA, siRNA targeting HcRed (nucleotides 99-117) was used (Dharmacon).

### Cell Culture

HeLa, HEK293, HEK293T, MCF-7, COS-7, SV40 immortalized MEFs, 35-8 cells (immortalized p53-null MEFs) and B16 F10.9 melanoma cells were grown as previously described (e.g., Bialik S., et. al., (2008), Mol. Cell. Proteomics, 7:1089-98). To induce autophagy by amino acid deprivation, cells were washed twice with PBS and then incubated with EBSS (Biological Industries) for various time-points as indicated in the text. Torin1 was a kind gift from DM Sabatini.

### In Vivo Phospho Labeling

In vivo labeling was achieved by growing HeLa cells on 15 cm plates in DMEM without sodium phosphate and sodium pyruvate (Gibco), supplemented with 10% FBS, for 3 h. 1 mCi of ³³-[P]-orthophosphate was then added for 2 h, and the protein was immunoprecipitated as described hereinbelow.

### Protein Analysis

Cell lysis and immunoblotting were done as previously described (e.g., Bialik S., et. al., (2008), Mol. Cell. Proteomics, 7:1089-98). Proteins were separated by SDS-PAGE and blotted onto nitrocellulose membranes, which were incubated with the following antibodies: monoclonal antibodies to p14ARF, Flag, α-tubulin and actin (Sigma), GFP (Roche) or DAP1 (Abcam) or polyclonal antibodies to LC3 (Sigma), mTOR, phospho-4E-BP1 (Thr 37/46), p70S6K and phospho-p70S6K (Thr389) (Cell Signaling). Polyclonal anti-phospho-Ser51 DAP1 antibody was raised in rabbits immunized with the phosphorylated peptide CEWESP(pS)PPKPT (wherein (pS) denotes a phosphorylated serine residue; SEQ ID NO: 18) (Bethyl laboratories). Detection was done with either HRP-conjugated goat anti-mouse or anti-rabbit secondary antibodies (Jackson ImmunoResearch), followed by enhanced chemiluminescence (SuperSignal, Pierce). Protein densitometric analysis was performed using NIH Imaging Software on scanned blots, with protein levels normalized to α-tubulin.

### GFP-LC3 Punctate Staining Assay

HeLa cells stably expressing GFP-LC3 were plated on 13 mm glass coverslips and subjected to starvation-induced autophagy as described above for 2-6 h. The cells were then fixed with 3.7% formaldehyde and viewed by fluorescent microscopy (Olympus BX41) with 60× (N.A. 1.25) UPlan-Fl oil immersion objectives, and digital images obtained with a DP50 CCD camera using ViewfinderLite and StudioLite software (Olympus).

### Immunoprecipitation and In Vitro Phosphatase Assay

Cells were washed with PBS and extracted in ice-cold lysis buffer as previously described (e.g., Bialik S., et. al., (2008), Mol. Cell. Proteomics, 7:1089-98). Protein extracts were pre-cleared with protein G or protein A-agarose beads (Santa Cruz) and then incubated with anti-FLAG M2 beads (Sigma) or with the beads pre-bound to the relevant antibodies. Immunoprecipitates were washed and the protein eluted from the beads with an excess of FLAG peptide. In order to dephosphorylate DAP1, the precipitated protein was washed with CIP buffer (NEB), and then incubated with the phosphatase (10 U) for 30 min at 37° C. Immunoprecipitation of mTOR was done as described previously (e.g., Sancak Y., et. al., (2007), Molecular cell, 25:903-15). Briefly, cells were lysed in ice-cold lysis buffer (40 mM HEPES [pH 7.4], 2 mM EDTA, 10 mM pyrophosphate, 10 mM glycerophosphate, 0.3% CHAPS, in the presence of protease inhibitors, and incubated with anti mTOR antibody (Santa Cruz) overnight at 4° C. After incubation with a protein A Sepharose for 1 h, immunoprecipitates were washed repeatedly with low salt wash buffer (40 mM HEPES [pH 7.4], 150 mM NaCl, 2 mM EDTA, 10 mM pyrophosphate, 10 mM glycerophosphate, 0.3% CHAPS).

### In Vitro Kinase Assay for Mtor Activity

For kinase assays, immunoprecipitates were washed three times in low salt wash buffer, followed by two additional washes in 25 mM HEPES (pH 7.4), 20 mM KCl. Kinase assays were performed for 20 min at 30° C. in a final volume of 30 l consisting of mTOR kinase buffer (25 mM HEPES [pH 7.4], 50 mM KCl, 10 mM MgCl₂, 250 μM ATP) and 1.5 μg bacterially produced His-DAP1 or 1 μg GST-4E-BP1 as the substrates. Torin1 was added to the reaction at a final concentration of 50 nM. Reactions were stopped by the addition of sample buffer and boiling for 5 min, and analyzed by SDS-PAGE and immunoblotting.

### RNA Analysis

Total cellular RNA was isolated with Trizol reagent (Invitrogen). Real-time RT-PCR was performed by first generating random primed cDNAs using the Superscript kit (Invitrogen). cDNA was then amplified by PCR in a light-Cycler⁴⁸⁰ (Roche) using SYBR Green I (Roche). Each primer pair was designed to span an intron. Primer pairs used were:

DAP1:
(SEQ ID NO: 8)
TGCGTCCTCGAAAAGC
and
(SEQ ID NO: 9)
GGCCTTGAAGGGTACAT;
HPRT:
(SEQ ID NO: 10)
TGACACTGGCAAAACAATGCA
and
(SEQ ID NO: 11)
GGTCCTTTTCACCAGCAAGCT.

### In Gel Proteolysis and Mass Spectrometry Analysis

Gel bands were excised and subjected to mass spectrometric analysis at the Smoler Proteomics Center at the Technion (Haifa, Israel). The proteins in the gel were reduced with 10 mM DTT, modified with 40 mM iodoacetamide and trypsinized (modified trypsin (Promega)) at a 1:100 enzyme-to-substrate ratio. The resulting tryptic peptides were resolved by reverse-phase chromatography on 0.1×200-mm fused silica capillaries (J&W, 100 micrometer ID) packed with Everest reversed phase material (Grace Vydac, Calif.). The peptides were eluted with linear 120 minute gradients of 5 to 95% of acetonitrile with 0.1% formic acid in water at flow rates of 0.4 μl/min. Mass spectrometry was performed by an ion-trap mass spectrometer (Orbitrap, Thermo) in a positive mode using repetitively full MS scans followed by collision induced dissociation (CID) of the 5 most dominant ions selected from the first MS scan. The mass spectrometric data was clustered and analyzed using the Sequest software (J. Eng and J. Yates, University of Washington and Finnigan, San Jose) and Pep-Miner (Beer, I., et. al., (2004), Proteomics, 4:950-60), searching against the human sequences within the NR—NCBI database.

### Bioinformatics Analysis

Sequences of DAP1 orthologs were extracted using protein Blast (NCBI), and culled to exclude predicted proteins, protein fragments, and redundancies. Pair-wise alignments were performed using Blast2Seq (Tatusova, T. A and Madden, T. L., (1999), FEMS microbiology letters, 174:247-50) and Bestfit from the GCG package (Wisconsin Package Version 10.3, Accelrys Inc., San Diego, Calif.). Multiple alignments were performed using ClustalW version 1.83 (Thompson J. D., et. al., (1994), Nucleic acids research, 22:4673-80) and visualized using Prettybox from the GCG package.

### Statistical Analysis

The statistical significance of differences between means was assessed by two-tailed Student's T-test. Values of p<0.05 were considered significant.

### Example 1

### DAP1 is a Highly Conserved Proline Rich Phosphoprotein

Human DAP1 gene encodes a single abundant mRNA transcript (2.4 Kb in size), which is ubiquitously expressed in many types of cells and tissues (Deiss, L. P., et. al., (1995), Genes & Development, 9:15-30). The predicted ORF of DAP1 corresponds to a small protein of 102 amino acids in length, rich in prolines (15%) and lacking any identifiable motifs (FIG. 1A). A single 17 kDa protein is translated in cells from the corresponding Flag-tagged cDNA, and endogenous DAP1 protein runs as a single 15 kDa protein (FIG. 2A). In vivo labeling of HeLa cells with ³³-[P]-orthophosphate followed by immunoprecipitation from cells revealed that DAP1 is a phosphoprotein in growing cells (FIG. 2B).

Through database searches, DAP1 orthologues were identified in most eukaryotes; several representative sequences are shown in FIG. 2C. Sequence alignment of these DAP1 orthologues using the ClustalW Program shows a high degree of conservation during evolution. As indicated in FIG. 2D, human DAP1 shows 97% similarity (96% identity) to the mouse DAP1 and shares 43% similarity with the C. elegans DAP1 sequence.

### Example 2

### Amino Acid Starvation Increases the Electrophoretic Mobility of DAP1 on Gels, Indicative of its Reduced Phosphorylation

Several stress conditions were applied to HeLa cells to identify a cellular setting in which DAP1 may be regulated. Unexpectedly, t following amino acid starvation, an increase in the steady state levels of the protein was observed, accompanied by an enhanced electrophoretic mobility on gels (FIGS. 1B and 1C). These two regulatory events characterizing the DAP1 protein response to amino acid deprivation were detected in all cell lines tested, including human (HeLa, MCF7, HEK293), monkey (COS-7) and mouse (B16, SV40 immortalized mouse embryonic fibroblasts (MEFs), 35-8) cell lines (FIG. 1D).

The increase in DAP1 protein levels occurred as early as 2-4-h after culturing HeLa cells in media lacking amino acids (EBSS), and persisted for at least 24 h (FIGS. 1B, 1C and 3A). The strong increase in protein levels was associated with only a small elevation in mRNA levels (FIGS. 1B and 1C), suggesting that additional post-transcriptional regulatory processes may take place as well. DAP1 is a stable protein with a turnover that exceeds 10 h (FIG. 3B), thus ruling out the possibility that the rapid increase in protein steady state levels results from protein stabilization. Notably, the increase in DAP1 protein takes place under conditions in which overall protein translation is suppressed (Dann, S. G., and Thomas, G. (2006), FEBS Lett, 580:2821-9; de Haro, C., et. al., (1996), Faseb J., 10:1378-87). As seen in FIG. 3C, the short-lived p14ARF protein declined at the same time in which DAP1 protein was up-regulated. Altogether, the data indicates that in contrast to the majority of cellular proteins, DAP1 protein translation continues and is even up-regulated in response to amino acid starvation.

The second level of DAP1 regulation, involves the apparent changes in its electrophoretic mobility on gels. The fast migrating form of DAP1, which appeared concomitant with the disappearance of the slow migrating form characteristic of nutrient rich conditions (FIGS. 1B and 1D), occurred as early as 0.5-2 h after shifting the cultures to EBSS, and persisted for the entire starvation period (FIG. 3A). The dynamics of the changes in DAP1 electrophoretic mobility are very rapid, as re-feeding of the starved cells with amino acids resulted in a fast upshift of the DAP1 band as early as 0.5-1 h post re-feeding (FIG. 1E; see also FIG. 4F). The identification of DAP1 as a phosphoprotein by in vivo phospho-labeling (FIG. 2B), suggests that the change in electrophoretic mobility may represent a change in the phosphorylation state of the protein. To test this, DAP1 was immunoprecipitated from cells that were either starved of amino acids (Starved) or grown in nutrient rich conditions (Unstarved) and the captured protein was treated with the generic Calf Intestinal Alkaline Phosphatase (CIP). Treatment of DAP1 immunoprecipitated from control unstarved cells with CIP resulted in the appearance of the fast migrating form, while the slow migrating form disappeared. Notably, this new band appeared at the same position as DAP1 immunoprecipitated from starved cells, with or without CIP treatment (FIG. 1F), suggesting that the faster form observed upon starvation is in fact dephosphorylated DAP1. Thus, DAP1 is phosphorylated in nutrient rich conditions and undergoes dephosphorylation upon amino acid starvation.

### Example 3

### Mapping DAP1 Phosphorylation Site/S

In order to map DAP1 phosphorylation site/s, stable cell lines of HEK293 cells that express DAP1 tagged with Flag at the C terminus (DAP1-Flag) were generated. Ectopically expressed DAP1-Flag retained its ability to undergo the electrophoretic mobility shift (FIG. 4A). DAP1-Flag was immunopurified from stably transfected polyclonal cell cultures using anti-FLAG M2 beads and either treated or not with CIP. After resolution on SDS-PAGE, the DAP1-Flag bands were excised and subjected to phosphopeptide mapping analysis by liquid chromatography-mass spectrometry/mass spectrometry (LC-MS/MS). Ser3 and Ser51 of DAP1 were identified as phosphorylated residues (FIG. 1A). In addition, the N terminal methionine was cleaved and Ser2 was modified by N-acetylation.

In order to identify the DAP1 residues that undergo dephosphorylation during starvation, the ratio of phosphorylated and non-phosphorylated peptides was compared in DAP1 immunoprecipitated from extracts of control unstarved cells vs. starved cells. This comparison revealed that Ser51 is dephosphorylated during starvation. These data are presented in FIGS. 4B and 4C; each figure represents the abundance of the different peptides isolated by the LC-MS/MS analysis of the different samples. Notably, technical difficulties prevented the detection of peptides within the N terminal one-third of DAP1 in the starved sample, and therefore the status of the phospho-site on Ser3 during starvation could not be evaluated by this approach.

Next, mutagenesis was used as a second independent strategy to map the phosphorylated residues that are modified by amino acid starvation. To this end, DAP1-Flag mutants in which the two Serines identified by LC-MS/MS were substituted to Ala or to Asp were generated and transfected into HEK293T cells, to determine whether one or more mutations might abolish the starvation-induced migration shift on gels. In fact, substitution of Ser3 to Ala or Asp (3 S/A or 3 S/D, respectively) abrogated the gel migration shift responses to starvation (FIG. 4D, upper panel). Furthermore, the non-phosphorylatible mutant (3 S/A) and the phospho-mimetic mutant (3 S/D) appeared at the same positions as the dephosphorylated and phosphorylated forms of the WT construct, respectively. These results indicate that Ser3 is also dephosphorylated upon starvation, and that the loss of the phosphate residue on this site is responsible for the migration shifts on gels. (Since these experiments were performed in transiently transfected cells, variations in steady state levels of the ectopically expressed proteins are not relevant and vary among experiments).

Notably, the substitution of Ser51 to Ala or Asp (51 S/A or 51 S/D, respectively) did not abolish the gel migration shift upon starvation (FIG. 4D, middle panel). Thus, the starvation-induced dephosphorylation of Ser51 detected by the MS analysis does not confer any conformational or electrostatic changes that can be detected by altered electrophoretic mobility. The double mutants, 3,51 SS/AA and 3,51 SS/DD, as expected, ran on gels in the same pattern as the single mutations of Ser3 (FIG. 4D, lower panel).

To further follow the fate of Ser51 phosphorylation in cells, anti-phospho-Ser51 antibodies were generated (Bethyl Laboratories). These antibodies recognize the ectopically expressed WT protein but fail to detect the 51 S/A mutant (FIG. 4E). At the endogenous level, amino acid starvation of HeLa cells resulted in a significant decrease in the phospho-Ser51 signal, while total DAP1 protein was markedly elevated at this time point (FIG. 4F), indicating by a second independent approach (in addition to the MS analysis) that endogenous DAP1 protein undergoes dephosphorylation on Ser51 during starvation.

### Example 4

### Identifying mTOR as the Specific DAP1 Kinase

Re-culturing the starved cells in complete medium (marked as Re-feeding) restored the phosphorylation on both sites, Ser3 (as detected by the return of the slowly migrating band) and Ser51 (as detected by anti-phospho-Ser51 antibodies) (FIG. 4F), demonstrating that the changes in DAP1 phosphorylation state are very dynamic. These rapid changes in DAP1 phosphorylation state during starvation/re-feeding experiments prompted the inventors to search for the specific kinase of DAP1. Surprisingly, DAP1 phosphorylation state correlated with the activity of mTOR in cells, and the dynamics of the phospho Ser3 and Ser51 is similar to other substrates of mTOR, such as p70S6K (FIG. 4F) and 4E-BP1 (data not shown). Interestingly, Ser3 and Ser51 phosphorylation sites fall within “proline-directed” motifs like those identified in 4E-BP1 (Gingras, A. C., et. al., (2001), Genes & development, 15:2852-64) (FIG. 1A), which are known to be phosphorylated by mTOR (Burnett, P. E., et. al., (1998), PNAS, 95:1432-7; Gingras A. C., et. al., (1999), Genes & development, 13:1422-37). As a first approach to determine whether DAP1 is phosphorylated by mTOR in vivo, HeLa cells were treated with Torin1, a specific ATP competitive inhibitor of mTOR (Thoreen, C. C., et. al., (2009), J. Biol. Chem., 284:8023-32) and the status of phospho Ser3 and Ser51 was monitored. The drug caused the typical shift of DAP1 to the fast migrating form indicative of dephosphorylation of Ser3, and a decline of the phospho-Ser51 signal using anti-phospho Ser51 antibodies (FIG. 4G), demonstrating that phosphorylation of both Ser3 and Ser51 sites were significantly attenuated. Phosphoryaltion of p70S6K, used as a positive control, was similarly affected by Torin1.

In vitro kinase assays were then conducted to assess whether mTOR directly phosphorylates DAP1. To this end, endogenous mTOR was immunoprecipitated from HEK293T cells and subjected to in vitro kinase assays using bacterially produced recombinant His-DAP1 or GST-4E-BP1 as substrates. FIG. 2G shows that mTOR phosphorylates His-DAP1 on Ser51 as detected by the anti-phospho-Ser51 antibody. The phosphorylation of GST-4E-BP1 by mTOR was monitored by using phospho Thr 37/46 antibody. Notably, the phosphorylation of both substrates was attenuated when the in vitro reaction was done in the presence of Torin1, proving that purified cellular mTOR directly phosphorylates DAP1.

### Example 5

### DAP1 Knockdown Increases Autophagy

To study the functional outcome of the knockdown of DAP1 on the process of autophagy that develops in response to amino acid deprivation, polyclonal and monoclonal HeLa cell lines stably expressing a GFP-LC3 fusion protein were generated. LC3, one of the autophagy (Atg) genes involved in autophagosome formation, associates with the autophagosome membrane in its lipidated form (LC3-PE), thus serving as a marker for autophagosomes (Kabeya, Y., et. al., (2000), Embo J, 19:5720-8; Mizushima N., et. al., (2001), J. Cell Biol., 152:657-68). These stable clones were tested for their response to amino acid starvation, and all showed the expected induction of fluorescent punctate indicative of autophagosome formation, as opposed to the diffuse localization observed in nutrient rich conditions (see FIGS. 5A and 6A).

DAP1 was knocked down by transfecting specific shRNA plasmids into the GFP-LC3 polyclonal stable clones before exposing the cells to EBSS. As shown in FIGS. 5A and 5B, during starvation there is a strong increase in the number of cells displaying punctate fluorescent staining per total GFP positive cells in DAP1 knockdown cells in comparison to shRNA control cells. Western blot analysis demonstrated that as early as 2 h after starvation, free GFP accumulated to a greater extent in cells lacking DAP1 (FIG. 5C). During starvation, autophagosomal intra-luminal GFP-LC3 is degraded in the autolysosomes, and free GFP accumulates, as it is relatively stable to lysosomal proteases. Thus, increased accumulation of free GFP in DAP1 knockdown cells reinforces the point that knockdown of DAP1 increases autophagic activity, rather than blocking later stages of autolysosome maturation or degradation, which can also lead to the accumulation of autophagosomes. Similar acceleration of autophagosome formation was obtained when the knockdown experiments were performed in a GFP-LC3 monoclonal stable cell line (clone 7) and with another DAP1 shRNA vector, to exclude off target effects. The number of cells with punctate GFP staining was increased in these cells lacking DAP1 with both RNAi constructs, although DAP1 shRNA II had higher basal autophagy (FIGS. 6A, and 6B; p<0.005, Student t-test, DAP1 shRNA in comparison to control shRNA during starvation).

These results were confirmed by western blotting for endogenous LC3, which, when conjugated to PE (LC3-II), migrates as a faster form on gels (at 16 kDa vs. 18 kDa, for the unconjugated LC3-I). The knockdown of DAP1 by either one of the two shRNAs caused a significant increase in the LC3-II/LC3-I ratio as detected at 2 and 4 h of amino acid deprivation (FIG. 7), indicating that DAP1 knockdown accelerated autophagosome formation. Inhibitors of lysosomal activity (E64d and pepstatin A) did not abrogate the stimulatory effect of DAP1 knockdown on autophagy, but rather contributed to a significant, further elevation in the GFP-LC3 punctate staining (p<0.03, Student t-test, DAP1 siRNA+inhibitors vs. DAP1 siRNA-inhibitors) (FIG. 5D). In addition, the ratio between LC3-II/LC3-I on blots was elevated in DAP1 depleted cells during starvation and increased even more when the lysosomal inhibitors were added (FIG. 5E). These results prove that the increased accumulation of autophagosomes in cells lacking DAP1 results from enhancement of autophagic activity rather than from blockage of late stages of the process.

In conclusion, the knockdown experiments using three different DAP1 RNAi constructs (2 shRNAs and one siRNA pool, see Experimental Procedures), several clones of GFP-LC3 stably expressing cells, and a few methods to monitor autophagy, suggest that DAP1 plays a suppressive role in the autophagy process.

### Example 6

### Dephosphorylation of DAP1 Activates the Suppressive Function of the Protein in Autophagy

In order to test whether the dephosphorylation of DAP1 activates or inactivates the autophagic suppressive effects, the wild type (WT) DAP1, phospho-mimetic (3,51 SS/DD) or the non phosphorylatible (3,51 SS/AA) mutants were introduced into GFP-LC3 transfectants in which the endogenous protein was knocked down using individual siRNA that targets the 3′UTR of DAP1 mRNA. These siRNAs, while effective against the endogenous DAP1, did not affect the ectopically expressed mutants which were generated from a DAP1 construct that lacks the 3′UTR (FIG. 8B). During starvation, the 3,51 SS/AA mutant blocked the increase in autophagosome accumulation observed upon knockdown of DAP1, while the 3,51 SS/DD mutant failed to restore the response to the level of the control siRNA transfected cells (FIG. 8A). (Note that statistical comparison of the various control siRNA expressing cells under starvation, including those expressing the non phosphorylatible mutant (3,51 SS/AA) (gray bars, right panel, starved) indicated no significant difference in autophagosome accumulation). Notably, the overexpressed wild type protein was also active in these experiments (FIG. 8A), as it underwent strong dephosphorylation. These results demonstrate that the dephosphorylated form of DAP1 is the active form that suppresses autophagy.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A method for the modulation of autophagy comprising altering the phosphorylation of DAP1.

2. The method of claim 1, wherein said DAP1 is human DAP1, or wherein said human DAP1 comprises the amino acid sequence as set forth in SEQ ID NO:1.

3. (canceled)

4. The method of claim 1, wherein altering the phosphorylation of DAP1 comprises altering the phosphorylation state of at least one serine residue selected from serine 3 and serine 51 of human DAP1

5. The method of claim 1, wherein the modulation of autophagy is an increase in autophagy.

6. The method of claim 5, comprising enhancing the phosphorylation of DAP1.

7. The method of claim 1, wherein the modulation of autophagy is a decrease in autophagy.

8. The method of claim 7, comprising reducing the phosphorylation of DAP1.

9. The method of claim 8, wherein reducing the phosphorylation of DAP1 comprises inactivating Ser/Thr mammalian target of rapamycin (mTOR).

10. A method for treating an autophagy associated disease or disorder in a subject comprising suppressing autophagy in a cell, the method comprises reducing the phosphorylation of DAP1.

11. The method of claim 10, wherein said DAP1 is human DAP1, or wherein said human DAP1 comprises the amino acid sequence as set forth in SEQ ID NO:1.

12. (canceled)

13. The method of claim 10, wherein reducing the phosphorylation of DAP1 comprises reducing the phosphorylation state of at least one serine residue selected from serine 3 and serine 51 of human DAP1, or wherein the phosphorylation of DAP1 is reduced by inactivating mTOR.

14. (canceled)

15. The method of claim 10, wherein the autophagy associated disease or disorder is selected from the group consisting of: cancer, a neurodegenerative disease or disorder, type II diabetes and myopathy.

16-18. (canceled)

19. A human DAP1 variant, comprising at least one serine residue substituted with a phospho-silencing residue, wherein the at least one serine residue is selected from serine 3 and serine 51 of human DAP1 (SEQ ID NO: 1).

20. The human DAP1 variant of claim 19, comprising a substitution selected from the group consisting of: a substitution of serine 3 and serine 51 of human DAP1 with phospho-silencing residues, a substitution of serine 3 of human DAP1 with a phospho-silencing residue, and a substitution of serine 51 of human DAP1 with a phospho-silencing residue.

21. The human DAP1 variant of claim 20, comprising the amino acid sequence selected from the group consisting of: the amino acid sequence as set forth in SEQ ID NO: 2, the amino acid sequence as set forth in SEQ ID NO: 3, and the amino acid sequence as set forth in SEQ ID NO: 4.

22-25. (canceled)

26. The human DAP1 variant of claim 19, wherein said phospho-silencing residue is selected from the group consisting of alanine, isoleucine, leucine, asparagine, lysine, methionine, phenylalanine, glutamine, tryptophan, glycine, valine, proline, arginine and histidine, or wherein the phospho-silencing residue is alanine.

27. (canceled)

28. The human DAP1 variant of claim 26 selected from the group consisting of SEQ ID NO: 13, SEQ ID NO: 14 and SEQ ID NO: 15.

29. The human DAP1 variant of claim 19, for use in treating an autophagy associated disease or disorder or for use in reducing or suppressing autophagy in a cell.

30. (canceled)

31. An isolated polynucleotide encoding the human DAP1 variant of claim 19.

32. A recombinant polynucleotide construct comprising the isolated polynucleotide according to claim 31 operably linked to a transcription regulating sequence.

33. An expression vector comprising the isolated polynucleotide according to claim 31.

34. The expression vector of claim 33, wherein said expression vector is a plasmid or a virus.

35. A host cell transfected with to the vector of claim 33.

36. A pharmaceutical composition comprising a therapeutically effective amount of an active agent selected from the group consisting of: (a) an isolated polypeptide comprising the amino acid sequence of a human DAP1 variant; (b) an isolated polynucleotide encoding a human DAP1 variant; (c) an expression vector comprising the isolated polynucleotide of (b); and (d) a host cell transfected with the expression vector of (c); further comprising a pharmaceutically acceptable carrier, wherein said human DAP1 variant comprises at least one serine residue selected from serine 3 and serine 51 of human DAP1 (SEQ ID NO: 1) substituted with a phospho-silencing residue.

37. A method for treating an autophagy associated disease or disorder in a subject in need thereof, comprising administering to the subject a pharmaceutical composition of claim 36, thereby treating the autophagy associated disease or disorder in said subject.

38. The method of claim 37, comprising administering to said subject a therapeutically effective amount of a recombinant polynucleotide construct comprising the isolated polynucleotide encoding a human DAP1 variant operably linked to a transcription regulating sequence.

39. The method of claim 37, wherein the autophagy associated disease or disorder is selected from the group consisting of: cancer, a neurodegenerative disease or disorder, type II diabetes and myopathy.

40-42. (canceled)

43. A method of reducing or suppressing autophagy in a target cell, comprising exposing the target cell to the pharmaceutical composition of claim 36 in an amount sufficient to reduce or suppress autophagy.


SEQUENCE LISTING (18 sequences)

SEQ ID NO: 1 - protein, 102 aa, Homo sapiens
MSSPPEGKLETKAGHPPAVKAGGMRIVQKHPHTGDTKEEKDKDDQEWESPSPPKPTVFISGVIARGDKDFPPAAAQVAHQKPHASMDKHPSPRTQHIQQPRK

SEQ ID NO: 2 - protein, 102 aa, Artificial Sequence
  Synthetic peptide
MSXPPEGKLETKAGHPPAVKAGGMRIVQKHPHTGDTKEEKDKDDQEWESPXPPKPTVFISGVIARGDKDFPPAAAQVAHQKPHASMDKHPSPRTQHIQQPRK

SEQ ID NO: 3 - protein, 102 aa, Artificial Sequence
  Synthetic peptide
MSXPPEGKLETKAGHPPAVKAGGMRIVQKHPHTGDTKEEKDKDDQEWESPSPPKPTVFISGVIARGDKDFPPAAAQVAHQKPHASMDKHPSPRTQHIQQPRK

SEQ ID NO: 4 - protein, 102 aa, Artificial Sequence
  Synthetic peptide
MSSPPEGKLETKAGHPPAVKAGGMRIVQKHPHTGDTKEEKDKDDQEWESPXPPKPTVFISGVIARGDKDFPPAAAQVAHQKPHASMDKHPSPRTQHIQQPRK

SEQ ID NO: 5 - protein, 102 aa, Artificial Sequence; identical to SEQ ID NO: 2
  Synthetic peptide

SEQ ID NO: 6 - protein, 102 aa, Artificial Sequence; identical to SEQ ID NO: 3
  Synthetic peptide

SEQ ID NO: 7 - protein, 102 aa, Artificial Sequence; identical to SEQ ID NO: 4
  Synthetic peptide

SEQ ID NO: 8 - protein, 16 aa, Artificial Sequence
  Synthetic peptide
TGCGTCCTCGAAAAGC

SEQ ID NO: 9 - protein, 17 aa, Artificial Sequence
  Synthetic peptide
GGCCTTGAAGGGTACAT

SEQ ID NO: 10 - protein, 21 aa, Artificial Sequence
  Synthetic peptide
TGACACTGGCAAAACAATGCA

SEQ ID NO: 11 - protein, 21 aa, Artificial Sequence
  Synthetic peptide
GGTCCTTTTCACCAGCAAGCT

SEQ ID NO: 12 - protein, 2549 aa, Homo sapiens
MLGTGPAAATTAATTSSNVSVLQQFASGLKSRNEETRAKAAKELQHYVTMELREMSQEESTRFYDQLNHHIFELVSSSDANERKGGILAIASLIGVEGGNATRIGRFANYLRNLLPSNDPVVMEMASKAIGRLAMAGDTFTAEYVEFEVKRALEWLGADRNEGRRHAAVLVLRELAISVPTFFFQQVQPFFDNIFVAVWDPKQAIREGAVAALRACLILTTQREPKEMQKPQWYRHTFEEAEKGFDETLAKEKGMNRDDRIHGALLILNELVRISSMEGERLREEMEEITQQQLVHDKYCKDLMGFGTKPRHITPFTSFQAVQPQQSNALVGLLGYSSHQGLMGFGTSPSPAKSTLVESRCCRDLMEEKFDQVCQWVLKCRNSKNSLIQMTILNLLPRLAAFRPSAFTDTQYLQDTMNHVLSCVKKEKERTAAFQALGLLSVAVRSEFKVYLPRVLDIIRAALPPKDFAHKRQKAMQVDATVFTCISMLARAMGPGIQQDIKELLEPMLAVGLSPALTAVLYDLSRQIPQLKKDIQDGLLKMLSLVLMHKPLRHPGMPKGLAHQLASPGLTTLPEASDVGSITLALRTLGSFEFEGHSLTQFVRHCADHFLNSEHKEIRMEAARTCSRLLTPSIHLISGHAHVVSQTAVQVVADVLSKLLVVGITDPDPDIRYCVLASLDERFDAHLAQAENLQALFVALNDQVFEIRELAICTVGRLSSMNPAFVMPFLRKMLIQILTELEHSGIGRIKEQSARMLGHLVSNAPRLIRPYMEPILKALILKLKDPDPDPNPGVINNVLATIGELAQVSGLEMRKWVDELFIIIMDMLQDSSLLAKRQVALWTLGQLVASTGYVVEPYRKYPTLLEVLLNFLKTEQNQGTRREAIRVLGLLGALDPYKHKVNIGMIDQSRDASAVSLSESKSSQDSSDYSTSEMLVNMGNLPLDEFYPAVSMVALMRIFRDQSLSHHHTMVVQAITFIFKSLGLKCVQFLPQVMPTFLNVIRVCDGAIREFLFQQLGMLVSFVKSHIRPYMDEIVTLMREFWVMNTSIQSTIILLIEQIVVALGGEFKLYLPQLIPHMLRVFMHDNSPGRIVSIKLLAAIQLFGANLDDYLHLLLPPIVKLFDAPEAPLPSRKAALETVDRLTESLDFTDYASRIIHPIVRTLDQSPELRSTAMDTLSSLVFQLGKKYQIFIPMVNKVLVRHRINHQRYDVLICRIVKGYTLADEEEDPLIYQHRMLRSGQGDALASGPVETGPMKKLHVSTINLQKAWGAARRVSKDDWLEWLRRLSLELLKDSSSPSLRSCWALAQAYNPMARDLFNAAFVSCWSELNEDQQDELIRSIELALTSQDIAEVTQTLLNLAEFMEHSDKGPLPLRDDNGIVLLGERAAKCRAYAKALHYKELEFQKGPTPAILESLISINNKLQQPEAAAGVLEYAMKHFGELEIQATWYEKLHEWEDALVAYDKKMDTNKDDPELMLGRMRCLEALGEWGQLHQQCCEKWTLVNDETQAKMARMAAAAAWGLGQWDSMEEYTCMIPRDTHDGAFYRAVLALHQDLFSLAQQCIDKARDLLDAELTAMAGESYSRAYGAMVSCHMLSELEEVIQYKLVPERREIIRQIWWERLQGCQRIVEDWQKILMVRSLVVSPHEDMRTWLKYASLCGKSGRLALAHKTLVLLLGVDPSRQLDHPLPTVHPQVTYAYMKNMWKSARKIDAFQHMQHFVQTMQQQAQHAIATEDQQHKQELHKLMARCFLKLGEWQLNLQGINESTIPKVLQYYSAATEHDRSWYKAWHAWAVMNFEAVLHYKHQNQARDEKKKLRHASGANITNATTAATTAATATTTASTEGSNSESEAESTENSPTPSPLQKKVTEDLSKTLLMYTVPAVQGFFRSISLSRGNNLQDTLRVLTLWFDYGHWPDVNEALVEGVKAIQIDTWLQVIPQLIARIDTPRPLVGRLIHQLLTDIGRYHPQALIYPLTVASKSTTTARHNAANKILKNMCEHSNTLVQQAMMVSEELIRVAILWHEMWHEGLEEASRLYFGERNVKGMFEVLEPLHAMMERGPQTLKETSFNQAYGRDLMEAQEWCRKYMKSGNVKDLTQAWDLYYHVFRRISKQLPQLTSLELQYVSPKLLMCRDLELAVPGTYDPNQPIIRIQSIAPSLQVITSKQRPRKLTLMGSNGHEFVFLLKGHEDLRQDERVMQLFGLVNTLLANDPTSLRKNLSIQRYAVIPLSTNSGLIGWVPHCDTLHALIRDYREKKKILLNIEHRIMLRMAPDYDHLTLMQKVEVFEHAVNNTAGDDLAKLLWLKSPSSEVWFDRRTNYTRSLAVMSMVGYILGLGDRHPSNLMLDRLSGKILHIDFGDCFEVAMTREKFPEKIPFRLTRMLTNAMEVTGLDGNYRITCHTVMEVLREHKDSVMAVLEAFVYDPLLNWRLMDTNTKGNKRSRTRTDSYSAGQSVEILDGVELGEPAHKKTGTTVPESIHSFIGDGLVKPEALNKKAIQIINRVRDKLTGRDFSHDDTLDVPTQVELLIKQATSHENLCQCYIGWCPFW

SEQ ID NO: 13 - protein, 102 aa, Artificial Sequence
  Synthetic peptide
MSAPPEGKLETKAGHPPAVKAGGMRIVQKHPHTGDTKEEKDKDDQEWESPAPPKPTVFISGVIARGDKDFPPAAAQVAHQKPHASMDKHPSPRTQHIQQPRK

SEQ ID NO: 14 - protein, 102 aa, Artificial Sequence
  Synthetic peptide
MSAPPEGKLETKAGHPPAVKAGGMRIVQKHPHTGDTKEEKDKDDQEWESPSPPKPTVFISGVIARGDKDFPPAAAQVAHQKPHASMDKHPSPRTQHIQQPRK

SEQ ID NO: 15 - protein, 102 aa, Artificial Sequence
  Synthetic peptide
MSSPPEGKLETKAGHPPAVKAGGMRIVQKHPHTGDTKEEKDKDDQEWESPAPPKPTVFISGVIARGDKDFPPAAAQVAHQKPHASMDKHPSPRTQHIQQPRK

SEQ ID NO: 16 - DNA, 19 bp, Artificial Sequence
  Synthetic oligonucleotide
gctggacacccgcccgccg

SEQ ID NO: 17 - DNA, 19 bp, Artificial Sequence
  Synthetic oligonucleotide
ttgtgcagaaacacccaca

SEQ ID NO: 18 - protein, 12 aa, Artificial Sequence
  Synthetic peptide
CEWESPXPPKPT